SEQUENCE LISTING (5 sequences)

SEQ ID NO: 1 - protein, 147 aa, Aspergillus oryzae
  misc_feature: hemoglobin domain
MPLSPEQIQLIKATVPVLQQHGTTITTVFYNNMLTAHPELNAVFNNANKVNGHQPRALAGALFAYASHIDDLGALGPAVELICNKHASLYIQPEQYQIVGKFLLEAMGEVLGDALTPEILDAWATAYWQLADLMIGREAELYKQADG

SEQ ID NO: 2 - protein, 147 aa, Aspergillus niger
  misc_feature: A.niger hemoglobin domain with an artificially introduced methionine at position 1
MPLTPEQIKIIKATVPVLQEYGTKITTAFYMNMSTVHPELNAVFNTANQVKGHQARALAGALFAYASHIDDLGALGPAVELICNKHASLYIQADEYKIVGKYLLEAMKEVLGDACTDDILDAWGAAYWALADIMINREAALYKQSQG

SEQ ID NO: 3 - protein, 455 aa, Aspergillus niger
  misc_feature: A.niger flavohemoglobin
MDRRVTIASQVAIAAVAGYCIYKAFNARQQQKSLKDAVPKSSDAPLTPEQIKIIKATVPVLQEYGTKITTAFYMNMSTVHPELNAVFNTANQVKGHQARALAGALFAYASHIDDLGALGPAVELICNKHASLYIQADEYKIVGKYLLEAMKEVLGDACTDDILDAWGAAYWALADIMINREAALYKQSQGWTNWRQFRISKKVPESDEITSFYLEPVDGKPLPAFRPGQYISVSVQVPEEDPQARQYSLSDTSRSDYYRISVKKETGLDPRAPGAKRHPGYVSNVLHDMIKEGDLIDVSHPYGDFFLSTAEATHPIVLLSAGVGMTPMMSILNTITKKSNRKIHFIHGSRTTEARAFKSHVQKLEKEIPNMQVTYFLSRPGDSDQLGVDYHHAGRIDLQKLDGPSHLYLDNPSTEYYICGPDTFMTQMEEALKAYGVGDDRIKMELFGTGGVPHN

SEQ ID NO: 4 - DNA, 1368 bp, Aspergillus niger
atggaccgtcgcgtcacaattgcatcccaagtcgccatcgctgccgtggctggctattgcatctacaaggcatttaacgctcgacagcaacaaaaatccctgaaggatgccgtgcccaagtcttccgatgctccgctcacaccagagcagatcaaaataatcaaggccacagtccctgttctacaagaatatggaaccaaaatcaccactgccttctacatgaacatgtcgaccgtgcatcctgagttgaatgctgtgttcaacactgccaaccaggtcaaaggccaccaagcacgtgcgctagctggtgcactgttcgcatatgcatcccacatcgacgacctcggggccctcggtcctgccgttgaattaatatgcaacaagcatgcatctctatacatccaagctgacgaatacaagatcgttggaaagtacctactggaagctatgaaggaggtacttggagatgcctgcactgatgatattctcgatgcgtggggtgccgcatactgggccctggccgacatcatgatcaaccgcgaagccgcactctacaagcaaagccagggctggaccaactggcgccaattccgcatttccaagaaggtgcccgagtcggacgagatcacctcgttctacttggaaccagtcgacggcaagccactgccagccttcaggccaggccagtatatctcggtgagcgtccaggttccagaggaagacccccaagctcgtcagtactcgctcagtgatacctctcgctcggattactatcgcatcagtgtcaagaaggaaacggggcttgatcctcgtgctccgggtgctaagagacaccccgggtacgtctccaacgttcttcatgacatgatcaaggagggcgatctcatcgatgtttctcatccctacggagacttcttcttgtccacagctgaagctacccacccgattgttcttctgtccgcgggtgtcggtatgacgcccatgatgtccatcctgaacaccatcactaagaaaagcaatcgcaagatccacttcattcatggatcccgtaccaccgaggctcgcgctttcaagagccacgttcaaaaactggaaaaggaaatccctaacatgcaggtgacctatttcctcagcaggccaggtgacagtgaccaactgggtgttgattaccaccacgctggacgaattgacctccagaaactcgatggaccatcccatctgtatctggacaacccttccacggaatactatatttgtggtcccgacacctttatgacgcagatggaggaggctttgaaggcttatggtgtgggcgatgaccggataaagatggagttgtttggtacaggtggtgttcctcataactag

SEQ ID NO: 5 - DNA, 536 bp, Aspergillus oryzae
atgccgctctcccctgaacaaatccagctcatcaaggccaccgtgccggtcctccagcagcatggcaccaccatcaccaccgtgttctacaataacatgctgacggcccaccccgagttgaacgccgtcttcaacaacgccaacaaggtgaacggccatcagccccgcgccctggccggtgccctctttgcctatgcctcgcatatcgatgacctgggagctcttgggcccgctgtcgagttaatctgcaacaagcacgcgtcgctgtatatccaacccgagcaataccagatcgtgggcaagtttctgttggaggccatgggcgaggtcctcggtgatgcgctgacccccgagatcctggacgcctgggccaccgcatactggcagcttgccgacctcatgatcggtcgtgaggccgaactgtacaagcaggccgacggatggacggacttccgccacttccgtgtcgccaagaaggtccctgagtcctcggagatcacctcgttctacctcgagcccgttgatggcaaagcccc

(19) United States
(12) Patent Application Publication
(10) Pub. No.: US 2008/0193969 A1
(43) Pub. Date: Aug. 14, 2008

(54) Hemoglobin Overexpression in Fungal Fermentations

(76) Inventors: Rob Te Biesebeke, Amersfoort (NL); Peter Jan Punt, Houten (NL); Cornelis Antonius Maria Jacobus Johannes Van den Hondel, Gouda (NL); Willem Meindert De Vos, Bennekon (NL)

Correspondence Address: NIXON & VANDERHYE, PC, 901 NORTH GLEBE ROAD, 11TH FLOOR, ARLINGTON, VA, 22203, US

(21) Appl. No.: 11/795,043

(22) PCT Filed: Jan. 12, 2006
(86) PCT No.: PCT/NL06/50006

§ 371 Date: Feb. 1, 2008

(30) Foreign Application Priority Data
Jan. 12, 2005 (EP) 05075097.5

(51) Int. Cl. C12P 35/00; C12N 1/00; C12P 1/02; C12P 19/00; C12P 7/42; C12P 7/50; C12P 7/44; C12P 7/46; C12P 21/04; C07K 14/00; C12N 15/11; C12P 7/56; C12P 13/04; C12P 7/48; C12P 7/64; C12P 7/06; C12P 23/00; C12P 17/00

(52) U.S. Cl. 435/47; 435/254.11; 435/254.3; 435/254.4; 435/254.5; 435/254.6; 435/254.7; 435/255.2; 435/255.4; 435/255.5; 435/171; 435/71.1; 435/72; 435/146; 435/143; 435/142; 435/145; 435/139; 435/106; 435/144; 435/134; 435/161; 435/117; 435/69.1; 536/23.1; 530/350

(57) ABSTRACT

The present invention relates to fungal host cells that are transformed with a nucleic acid construct encoding a fungal oxygen-binding proteins or fragments thereof that comprise the oxygen-binding domain. Upon transformation of the host cell with the construct, the oxygen-binding protein confers to the host cell improved fermentation characteristics as compared to untransformed host cells. These characteristics include e.g. increases in oxygen uptake rates, biomass densities, volumetric productivities and/or product yields. The invention further relates to fermentation processes in which the host cells are used and to fungal oxygen binding proteins, in particular fungal flavohemoglobins and hemoglobin domains, and to nucleotides sequences encoding these proteins.

### FIELD OF THE INVENTION

The present invention relates to fungi that overexpress fungal oxygen-binding proteins, particularly (flavo)hemoglobins, to improve the fermentation characteristics of the fungi during solid state as well as submerged fermentation processes. The invention further relates to fermentation processes in which these fungi are applied, and to fungal oxygen-binding proteins, nucleic acids encoding these proteins and vectors comprising such nucleic acids.

### BACKGROUND OF THE INVENTION

Oxygen is essential for maximal energy yield and optimal utilization of substrate in every aerobic organism (Frey and Kalio 2003). During growth of A. oryzae on solid substrates, the aerial hyphae account for 70% of the oxygen uptake (Rahardjo et al., 2001). It is shown that diffusion of oxygen is limited in the filamentous fungal layer that covers the solid substrate and that the substrate penetrative byphae are limited in oxygen consumption and growth (Oostra et al., 2001a, Rahardjo et al., 2001). Therefore oxygen supply to microbial cells that are in close contact with the substrate is considered as a bottleneck in solid-state fermentation (Thibault et al., 2000, Oostra et al., 2001a).

Hemoglobins bind O₂ reversibly and have been discovered in a wide range of organisms including vertebrates, invertebrates, higher plants, fungi and bacteria (Weber and Vinogradov, 2001). Despite the fact that all known hemoglobins have a highly variable primary amino acid sequence they all show a 6 to 8 alpha helical arrangement that facilitates binding of heme in the hydrophobic core of the protein (Frey and Kalio 2003). Hemoglobins bridge a wide variation in O₂ tensions at the sites of O₂ loading and unloading and therefore play a major role in O₂ transport although specific hemoglobins may be specialized for particular functions (Weber and Vinogradov 2001).

The expression of Vitreoscilla hemoglobin in Eschericia coli (Yu et al., 2002, Andersson et al., 2003) and Enterobacter aerogenes (Geckil et al., 2003) has been shown to correlate with improved protein synthesis, enhanced intracellular ribosome and tRNA contents and improved growth/survival properties. Moreover, Vitreoscilla hemoglobin expression in Yarrowia lipolitica (Bhave and Chattoo 2003), Pichia pastoris (Wu et al., 2003), and Acremonium chrysogenum (DeModena et al., 1993) resulted in higher enzyme production, improved growth and higher cephalosporin C production. Expression of Vitreoscilla hemoglobin in Aspergillus terreus resulted in improved itaconic acid production (Lin et al., 2004).

Flavohemoglobins (FlavoHb) consist of an amino-terminal hemoglobin domain that reversibly binds oxygen and a carboxy-terminal redox active domain with putative binding sites for NAD(P)H and FAD. FlavoHbs have been described for a number of bacterial taxons and several fungal species like Saccharomyces cerevisiae (Zhu and Riggs 1992), Fusarium oxysporum (Takaya et al., 1997), Candida norvegensis (Kobayashi et al., 2002) and Cryptococcus neoformans (Jesus-Berrios et al., 2003). FlavoHbs appear to provide protection to nitrosative (NO) stress in bacteria (reviewed by Frey and Kallio 2003). Also in fungi the involvement in protection against nitrosative stress is suggested. After deletion of the S. cerevisiae flavoHb (YHB1) gene and exposure to an artificial NO donor, higher levels of nitrosylation of high molecular mass molecules were measured compared to the wild-type (Liu et al., 2000). The flavoHb of C. neoformans an established human fungal pathogen that replicates in macrophages protects from nitrosative stress and is necessary for full pathogenesis (Jesus-Berrios et al 2003). Other studies have suggested a role of the S. cerevisiae Yhblp in protection against oxidative stress (Zhao et al., 1996, Buisson and Labbe-Bois 1998). In contrast to bacterial flavoHb's, the high affinity of oxygen binding of Candida norvegensis flavoHb led Kobayashi et al., (2002) to suggest that yeast flavoHb could also serve as an oxygen storage protein.

Fungal flavoHbs or the hemoglobin domains thereof have however not yet been used for improvement of fermentation properties of fungal production organisms. It is thus an object of the present invention to provide for nucleic acid sequences encoding novel fungal flavoHbs and hemoglobin domains for overexpression in fungi that are used as production organisms in fermentation processes. A particular object of the present invention is to provide for self-cloning strategies for fungi, include filamentous fungi like Aspergillus, in which fungal flavoHb and hemoglobin domain genes are used instead of e.g. the bacterial Vitreoscilla gene to provide for industrial fungal production strains with improved fermentation characteristics.

### DESCRIPTION OF THE INVENTION

### Definitions

The term “gene” means a DNA fragment comprising a region (transcribed region), which is transcribed into an RNA molecule (e.g. an mRNA) in a cell, operably linked to suitable regulatory regions (e.g. a promoter). A gene may thus comprise several operably linked fragments, such as a promoter, a 5′ leader sequence, a coding region and a 3′nontranslated sequence (3′ end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide or which is active itself (e.g. in posttranscriptional gene silencing or RNAi). In one embodiment the 5′-end of the coding sequence preferably encodes a (homologous or heterologous) secretion signal, so that the encoded protein or peptide is secreted out of the cell. The coding sequence is preferably in sense-orientation and encodes a desired, biologically active protein or protein fragment.

A “chimeric” (or recombinant) gene refers to any gene, which is not normally found in nature in a species, in particular a gene in which different parts of the nucleic acid region are not associated in nature with each other. For example the promoter is not associated in nature with part or all of the transcribed region or with another regulatory region. The term “chimeric gene” is understood to include expression constructs in which a promoter or transcription regulatory sequence is operably linked to one or more coding sequences or to an antisense (reverse complement of the sense strand) or inverted repeat sequence (sense and antisense, whereby the RNA transcript forms double stranded RNA upon transcription).

The term “nucleic acid sequence” (or nucleic acid molecule) refers to a DNA or RNA molecule in single or double stranded form, particularly a DNA encoding a protein or protein fragment according to the invention. An “isolated nucleic acid sequence” refers to a nucleic acid sequence which is no longer in the natural environment from which it was isolated, e.g. the nucleic acid sequence in a bacterial host cell or in the plant nuclear or plastid genome.

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules.

The term peptide herein refers to any molecule comprising a chain of amino acids that are linked in peptide bonds. The term peptide thus includes oligopeptides, polypeptides and proteins, including multimeric proteins, without reference to a specific mode of action, size, 3-dimensional structure or origin. A “fragment” or “portion” of a protein may thus still be referred to as a “protein”. An “isolated protein” is used to refer to a protein which is no longer in its natural environment, for example in vitro or in a recombinant (fungal) host cell. The term peptide also includes post-expression modifications of peptides, e.g. glycosylations, acetylations, phosphorylations, and the like.

A “truncated protein” refers herein to a protein which is reduced in amino acid length compared to the wild type protein. Especially, certain domains may be absent, e.g. in a flavohemoglobin the redox active domain with potential binding sites for NAD(P)H and FAD may be absent. In a preferred embodiment a truncated flavohemoglobin lacks the redox active domain with potential binding sites for NAD(P)H and FAD but retains the hemoglobin domain.

A “chimeric protein” or “hybrid protein” is a protein composed of various protein “domains” (or motifs) which is not found as such in nature but which a joined to form a functional protein, which displays the functionality of the joined domains (for example receptor binding). A chimeric protein may also be a fusion protein of two or more proteins occurring in nature. The term “domain” as used herein means any part(s) or domain(s) of the protein with a specific structure or function that can be transferred to another protein for providing a new hybrid protein with at least the functional characteristic of the domain.

The term “expression vector” refers to nucleotide sequences that are capable of effecting expression of a gene in host cells or host organisms compatible with such sequences. These expression vectors typically include at least suitable transcription regulatory sequences and optionally, 3′ transcription termination signals. Additional factors necessary or helpful in effecting expression may also be present, such as expression enhancer elements. DNA encoding the polypeptides of the present invention will typically be incorporated into the expression vector. The expression vector will be introduced into a suitable host cell and be able to effect expression of the coding sequence in an in vitro cell culture of the host cell. The expression vector preferably is suitable for replication in a fungal host cell or in a prokaryotic host.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer. A “tissue specific” promoter is only active in specific types of tissues or cells.

The term “selectable marker” is a term familiar to one of ordinary skill in the art and is used herein to describe any genetic entity which, when expressed, can be used to select for a cell or cells containing the selectable marker. Selectable markers may be dominant or recessive or bidirectional. The selectable marker may be a gene coding for a product which confers antibiotic resistance to a cell expressing the gene or a non-antibiotic marker gene, such as a gene relieving other types of growth inhibition, i.e. a marker gene which allow cells containing the gene to grow under otherwise growth-inhibitory conditions. Examples of such genes include a gene which confers prototrophy to an auxotrophic strain, e.g. dal genes introduced in a dal.sup.-strain (cf. B. Diderichsen in Bacillus: Molecular Genetics and Biotechnology Applications, A. T. Ganesan and J. A. Hoch, Eds., Academic Press, 1986, pp. 35-46) or a thy gene introduced in a thy.sup.-cell (cf. Gryczan and Dubnau (1982), Gene, 20, 459-469) or a gene which enables a cell harbouring the gene to grow under specific conditions such as an amdS gene, the expression of which enables a cell harbouring the gene to grow on acetamide as the only nitrogen or carbon source (e.g. as described in EP 635 574), or a gene which confers resistance towards a heavy metal (e.g. arsenite, arsenate, antimony, cadmium or organo-mercurial compounds) to a cell expressing the gene. Cells surviving under these conditions will either be cells containing the introduced DNA construct in an extrachromosomal state or cells in which the above structure has been integrated. Alternatively, the selectable marker gene may be one conferring immunity to a cell expressing the gene. The term “reporter” may be used interchangeably with marker, although it is mainly used to refer to visible markers, such as green fluorescent protein (GFP).

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

The term “ortholog” of a gene or protein refers herein to the homologous gene or protein found in another species, which has the same function as the gene or protein, but is (usually) diverged in sequence from the time point on when the species harbouring the genes diverged (i.e. the genes evolved from a common ancestor by specification).

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. In this context, the use of only “homologous” sequence elements allows the construction of “self-cloned” organisms:

“Self-cloning” is defined herein as in European Directive 98/81/EC Annex II: Self-cloning consists in the removal of nucleic acid sequences from a cell of an organism which may or may not be followed by reinsertion of all or part of that nucleic acid (or a synthetic equivalent) with or without prior enzymic or mechanical steps, into cells of the same species or into cells of phylogenetically closely related species which can exchange genetic material by natural physiological processes where the resulting micro-organism is unlikely to cause disease to humans, animals or plants. Self-cloning may include the use of recombinant vectors with an extended history of safe use in the particular micro-organisms.

When used to indicate the relatedness of two nucleic acid sequences the term “homologous” means that one single-stranded nucleic acid sequence may hybridise to a complementary single-stranded nucleic acid sequence. The degree of hybridisation may depend on a number of factors including the amount of identity between the sequences and the hybridisation conditions such as temperature and salt concentration as discussed later.

The term “substantially identical”, “substantial identity” or “essentially similar” or “essential similarity” means that two peptide or two nucleotide sequences, when optimally aligned, such as by the programs GAP or BESTFIT using default parameters, share at least a certain percentage of sequence identity as defined elsewhere herein. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). It is clear that when RNA sequences are said to be essentially similar or have a certain degree of sequence identity with DNA sequences, thymine (T) in the DNA sequence is considered equal to uracil (U) in the RNA sequence.

Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA or the open-source software Emboss for Windows (current version 2.7.1-07). Alternatively percent similarity or identity may be determined by searching against databases such as FASTA, BLAST, etc.

Optionally, in determining the degree of “amino acid similarity”, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

Nucleotide sequences encoding flavohemoglobins or hemoglobin domains of the invention may also be defined by their capability to “hybridize” with the nucleotide sequences of SEQ ID NO. 3 or SEQ ID NO. 4, under moderate, or preferably under stringent hybridisation conditions. “Stringent hybridisation” conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity.

“Moderate conditions” are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 90%.

“Fungi” are herein defined as eukaryotic microorganisms and include all species of the subdivision Eumycotina (Alexopoulos, C. J., 1962, In: Introductory Mycology, John Wiley & Sons, Inc., New York). The term fungus thus includes both filamentous fungi and yeast. “Filamentous fungi” are herein defined as eukaryotic microorganisms that include all filamentous forms of the subdivision Eumycotina and Oomycota (as defined by Hawksworth et al., 1995, supra). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. Filamentous fungal strains include, but are not limited to, strains of Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, and Trichoderma. “Yeasts” are herein defined as eukaryotic microorganisms and include all species of the subdivision Eumycotina that predominantly grow in unicellular form. Yeasts may either grow by budding of a unicellular thallus or may grow by fission of the organism.

The term “fungal”, when referring to a protein or nucleic acid molecule thus means a protein or nucleic acid whose amino acid or nucleotide sequence, respectively, naturally occurs in a fungus.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### DETAILED DESCRIPTION OF THE INVENTION

In a first aspect, the present invention relates to a fungal host cell transformed with a nucleic acid construct comprising a nucleotide sequence encoding an oxygen-binding protein. The oxygen-binding protein preferably is fungal oxygen-binding protein or a fragment thereof that comprises an oxygen-binding domain like e.g. a hemoglobin domain. Preferably the oxygen-binding protein is a flavohemoglobin or a fragment of a flavohemoglobin that comprises a hemoglobin domain. Preferably, the flavohemoglobin is a fungal flavohemoglobin and the fragment is a fragment of a fungal flavohemoglobin. More preferably, in the host cells of the invention the oxygen-binding proteins and fragments thereof are from a fungus selected from Aspergillus, Trichoderma, Humicola, Acremonium, Fusarium, Rhizopus, Mortierella, Penicillium, Myceliophthora, Chrysosporium, Mucor, Sordaria, Neurospora, Podospora, Monascus, Agaricus, Pycnoporus, Schizophylum, Trametes, Phanerochaete, Saccharomyces, Kluyveromyces, Candida, Pichia, Schizosaccharomyces, Hansenula, Kloeckera, Schwanniomyces, and Yarrowia.

Preferably, in the host cells of the invention, the nucleic acid construct upon transformation of the host cell, confers to the host cell an increase in a fermentation parameter compared to an otherwise identical host cell that is not transformed with the construct, whereby preferably both the transformed and untransformed host cells are grown under identical conditions. Preferably, the fermentation parameter is at least one of: (a) oxygen uptake rate; (b) biomass density; (c) volumetric productivity; and, (d) yield coefficient of fermentation product produced over substrate. The (specific) oxygen uptake rate is the amount of oxygen (grams or moles) consumed per time unit (hour) per amount of biomass (grams). Volumetric productivity is understood to mean the amount of product produced per time unit per unit fermenter volume and may be expressed as units or grams product per hour per liter fermenter or culture volume. The yield coefficient of fermentation product produced over substrate (Y_{PS}) may be expressed as units or grams of product produced per gram of substrate used. Alternatively it may be expressed on a C-molar basis, which is herein understood to mean the amount carbon atoms in product produced per the amount of carbon atoms in substrate utilised.

In a preferred host cell, at least one fermentation parameter of the transformed host cell is increased by at least 5, 10, 20, 50, 100, 200, or 500% as compared to the untransformed host cell.

The improved fermentation characteristics of the transformed host cells of the invention are the result of a higher steady state level of oxygen binding proteins in the transformed host cell as compared to an untransformed host cell. The steady state level of the oxygen-binding protein in a host cell may be expressed as the specific amount or activity of oxygen binding proteins. The specific amount or activity of oxygen-binding protein in the host cell is herein defined as the amount or activity of oxygen-binding protein per mg protein. The activity of oxygen-binding protein may be determined as described in Example 2.1.4. Preferably, transformation of a host cell with a nucleic acid construct of the invention confers to the host cell a specific amount or activity of oxygen-binding protein that is at least 5, 10, 20, 50, 100, 200, or 500% higher than in an otherwise identical untransformed host cell.

Preferably in a host cell according to the invention, the nucleotide sequence is selected from: (a) nucleotide sequences encoding a polypeptide comprising an amino acid sequence that has at least 49, 50, 51, 52, 55, 60, 70, 80, 90, 95, 98% sequence identity with the amino acid sequence of SEQ ID NO. 1 or 2; (b) nucleotide sequences the complementary strand of which hybridise to a nucleic acid molecule sequence of (a); and, (c) nucleotide sequences the sequence of which differs from the sequence of a nucleic acid molecule of (b) due to the degeneracy of the genetic code.

In a preferred embodiment, the nucleic acid construct used to transform a host cell according to the invention comprises a nucleotide sequence that encodes an amino acid sequence that naturally occurs in cells of the same species as the host cell or in cells of phylogenetically closely related species which can exchange genetic material by natural physiological processes, such that the transformed host cell is unlikely to cause disease to humans, animals or plants. Therefore preferably the amino acid sequence has at least 90% amino acid identity with the amino acid sequence of a fungal flavohemoglobin that naturally occurs in the host or with the amino acid sequence of a fragment of the flavohemoglobin comprising the hemoglobin domain. More preferably, the amino acid sequence identity is at least 95, 98, or 99%. Yet more preferably the amino acid sequence identity is 100%, i.e. the protein comprising the amino acid sequence of the flavohemoglobin or the hemoglobin domain is homologous to the host. Most preferably, also the nucleotide sequence encoding a polypeptide has 100% identity with the nucleotide sequence encoding a fungal flavohemoglobin that naturally occurs in the host or with the nucleotide sequence encoding a fragment of the flavohemoglobin comprising the hemoglobin domain, i.e. the nucleotide sequence is homologous to the host.

In a preferred host cell of the invention, the fragment comprising the hemoglobin domain comprises no more than 30, 15, 8, or 4 additional amino acids onto the N-terminus of the domain. Preferably the fragment comprising the hemoglobin domain comprises no more than 30, 15, 8, or 4 additional amino acids onto the C-terminus of the domain. Preferably, the domain comprises no more than 30, 15, 8, or 4 additional amino acids onto either terminus of the domain. The hemoglobin domain is herein defined as a polypeptide consisting of an amino acid sequence that has at least 49% sequence identity with the amino acid sequence of SEQ ID NO. 1 or 2 (and that is preferably aligned as depicted in FIG. 5B) and that has the ability to confer to a fungal host cell an increase in a fermentation parameter of at least 5% compared to an otherwise identical fungal host cell that is not transformed with the construct, whereby preferably both the transformed and untransformed host cells are grown under identical conditions, and whereby the fermentation parameter is at least one of: (a) oxygen uptake rate; (b) biomass density; (c) volumetric productivity; and, (d) yield coefficient of fermentation product produced over substrate. Most preferably, the domain comprises no additional amino acids and thus consists of an amino acid sequence that has at least 49% sequence identity with the amino acid sequence of SEQ ID NO. 1 or 2 and that is preferably aligned as depicted in FIG. 5B. The skilled person will appreciate that if in a fragment comprising a hemoglobin domain the first N-terminal amino acid is not methionine (as is e.g. the case with the hemoglobin domain of A. niger, SEQ ID NO. 2), the nucleotide sequence encoding the fragment may be engineered to replace the first N-terminal amino acid by a methionine or to have it preceded by a methionine.

The host cells according to the invention are preferably fungal host cell whereby a fungus is defined as herein above. Preferred fungal host cells are fungi that are used in industrial fermentation processes for the production of fermentation products as described below. A large variety of filamentous fungi as well as yeasts are use in such processes. Preferred filamentous fungal host cells may be selected from the genera: Aspergillus, Trichoderma, Humicola, Acremonium, Fusarium, Rhizopus, Mortierella, Penicillium, Myceliophthora, Chrysosporium, Mucor, Sordaria, Neurospora, Podospora, Monascus, Agaricus, Pycnoporus, Schizophylum, Trametes and Phanerochaete. Preferred fungal strains that may serve as host cells, e.g. as reference host cells for the comparison of fermentation characteristics of transformed and untransformed cells, include e.g. Aspergillus niger CBS120.49, CBS 513.88, Aspergillus oryzae ATCC16868, ATCC 20423, IFO 4177, ATCC 1011, ATCC 9576, ATCC14488-14491, ATCC 11601, ATCC12892, Aspergillus fumigatus AF293 (CBS101355), P. chrysogenum CBS 455.95, Penicillium citrinum ATCC 38065, Penicillium chrysogenum P2, Acremonium chrysogenum ATCC 36225, ATCC 48272, Trichoderma reesei ATCC 26921, ATCC 56765, ATCC 26921, Aspergillus sojae ATCC11906, Chrysosporium lucknowense ATCC44006 and derivatives of all of these strains. Particularly preferred as filamentous fungal host cell are Aspergillus niger CBS 513.88 and derivatives thereof. Preferred yeast host cells may be selected from the genera: Saccharomyces, Kluyveromyces, Candida, Pichia, Schizosaccharomyces, Hansenula, Kloeckera, Schwanniomyces, and Yarrowia. Optionally, the host cell of the invention comprises an elevated unfolded protein response (UPR) compared to the wild type cell to enhance production abilities of a polypeptide of interest. UPR may be increased by techniques described in US2004/0186070A1 and/or US2001/0034045A1 and/or WO01/72783A2 and/or WO2005/123763. More specifically, the protein level of HAC1 and/or IRE1 and/or PTC2 has been modulated, and/or the SEC61 protein has been engineered in order to obtain a host cell having an elevated UPR. Alternatively, or in combination with an elevated UPR, the host cell is genetically modified to obtain a phenotype displaying lower protease expression and/or protease secretion compared to the wild-type cell in order to enhance production abilities of a polypeptide of interest. Such phenotype may be obtained by deletion and/or modification and/or inactivation of a transcriptional regulator of expression of proteases. Such a transcriptional regulator is e.g. prtT. Lowering expression of proteases by modulation of prtT may be performed by techniques described in US2004/0191864A1. Alternatively, or in combination with an elevated UPR and/or a phenotype displaying lower protease expression and/or protease secretion, the host cell displays an oxalate deficient phenotype in order to enhance the yield of production of a polypeptide of interest. An oxalate deficient phenotype may be obtained by techniques described in WO2004/070022A2. Alternatively, or in combination with an elevated UPR and/or a phenotype displaying lower protease expression and/or protease secretion and/or oxalate deficiency, the host cell displays a combination of phenotypic differences compared to the wild cell to enhance the yield of production of the polypeptide of interest. These differences may include, but are not limited to, lowered expression of glucoamylase and/or neutral alpha-amylase A and/or neutral alpha-amylase B, protease, and oxalic acid hydrolase. Said phenotypic differences displayed by the host cell may be obtained by genetic modification according to the techniques described in US2004/0191864A1.

Host cells of the invention are transformed with a nucleic acid construct as further defined below and may comprise a single but preferably comprises multiple copies of the nucleic acid construct. The nucleic acid construct may be maintained episomally and thus comprise a sequence for autonomous replication, such as an ARS sequence. Suitable episomal nucleic acid constructs may e.g. be based on the yeast 2μ or pKD1 (Fleer et al., 1991, Biotechnology 9: 968-975) plasmids. Preferably, however, the nucleic acid construct is integrated in one or more copies into the genome of the host cell. Integration into the host cell's genome may occur at random by illegitimate recombination but preferably nucleic acid construct is integrated into the host cell's genome by homologous recombination as is well known in the art of fungal molecular genetics (see e.g. WO 90/14423, FP-A-0 481 008, EP-A-0 635 574 and U.S. Pat. No. 6,265,186).

A host cell of the invention may comprise further genetic modifications such as e.g. modifications that result in increased heme biosynthesis as e.g. described in U.S. Pat. No. 6,100,057.

Transformation of host cells with the nucleic acid constructs of the invention and additional genetic modification of the fungal host cells of the invention as described above may be carried out by methods well known in the art. Such methods are e.g. known from standard handbooks, such as Sambrook and Russel (2001) “Molecular Cloning: A Laboratory Manual (3^{rd} edition), Cold Spring Harbor Laboratory, Cold Spring Harbor Laboratory Press, or F. Ausubel et al, eds., “Current protocols in molecular biology”, Green Publishing and Wiley Interscience, New York (1987). Methods for transformation and genetic modification of fungal host cells are known from e.g. EP-A-0 635 574, WO 98/46772, WO 99/60102 and WO 00/37671.

In another aspect the invention relates to a nucleic acid construct comprising a nucleotide sequence encoding a oxygen-binding protein or fragment thereof as defined above and used for transformation of a host cell as defined above. In the nucleic acid construct, the nucleotide sequence encoding the oxygen-binding protein preferably is operably linked to a promoter for control and initiation of transcription of the nucleotide sequence in a host cell as defined below. The promoter preferably is capable of causing sufficient expression of the oxygen-binding protein in the host cell, to confer to the host cell an increased fermentation parameter as defined above. Preferably, the promoter causes an increase of the specific amount or activity of oxygen binding proteins in the transformed host cell as compared to an untransformed host cell as defined above. Promoters useful in the nucleic acid constructs of the invention include both constitutive and inducible natural promoters as well as engineered promoters. Promotors suitable to drive expression of the oxygen-binding proteins in the hosts of the invention include e.g. promoters from glycolytic genes (e.g. from a glyceraldehyde-3-phosphate dehydrogenase gene), ribosomal protein encoding gene promoters, alcohol dehydrogenase promoters (ADH1, ADH4, and the like), promoters from genes encoding amylo- or cellulolytic enzymes (glucoamylase, TAKA-amylase and cellobiohydrolase) Preferred promoters for the use in filamentous fungi are promoters obtained from the genes encoding A. oryzae TAKA amylase, Rhizomucor miehei aspartic proteinase, A. niger neutral alpha-amylase, A. niger acid stable alpha-amylase, A. niger or A. awamori glucoamylase (glaA), R. miehei lipase, A. oryzae alkaline protease, A. oryzae triose phosphate isomerase, A. nidulans acetamidase, the NA2-tpi promoter (a hybrid of the promoters from the genes encoding A. niger neutral alpha-amylase and A. oryzae triose phosphate isomerase), and mutant, truncated, and hybrid promoters thereof. Other preferred promoters for use in filamentous fungal cells are a promoter, or a functional part thereof, from a protease gene; e.g., from the F. oxysporum trypsin-like protease gene (U.S. Pat. No. 4,288,627), A. oryzae alkaline protease gene(alp), A. niger pacA gene, A. oryzae alkaline protease gene, A. oryzae neutral metalloprotease gene, A. niger aspergillopepsin protease pepA gene, or F. venenatum trypsin gene, A. niger aspartic protease pepB gene. Other promoters, both constitutive and inducible and enhancers or upstream activating sequences will be known to those of skill in the art. The promoters used in the nucleic acid constructs of the present invention may be modified, if desired, to affect their control characteristics. Preferably, the promoter used in the nucleic acid construct for expression of the oxygen-binding protein is homologous to the host cell in which the oxygen-binding protein is expressed.

In the nucleic acid construct, the 3′-end of the nucleotide acid sequence encoding the oxygen-binding protein preferably is operably linked to a transcription terminator sequence. Preferably the terminator sequence is operable in a host cell of choice, such as e.g. the yeast species of choice. In any case the choice of the terminator is not critical; it may e.g. be from any yeast gene, although terminators may sometimes work if from a non-yeast, eukaryotic, gene. Preferred terminators for filamentous fungal cells are obtained from the genes encoding A. oryzae TAKA amylase, A. niger glucoamylase (glaA), A. nidulans anthranilate synthase, A. niger alpha-glucosidase, trpC gene and Fusarium oxysporum trypsin-like protease. The transcription termination sequence further preferably comprises a polyadenylation signal. Preferred polyadenylation sequences for filamentous fungal cells are obtained from the genes encoding A. oryzae TAKA amylase, A. niger glucoamylase, A. nidulans anthranilate synthase, Fusarium oxyporum trypsin-like protease and A. niger alpha-glucosidase.

Optionally, a selectable marker may be present in the nucleic acid construct. As used herein, the term “marker” refers to a gene encoding a trait or a phenotype which permits the selection of, or the screening for, a host cell containing the marker. The marker gene may be an antibiotic resistance gene whereby the appropriate antibiotic can be used to select for transformed cells from among cells that are not transformed. Examples of suitable antibiotic resistance markers include e.g. dihydrofolate reductase, hygromycin-B-phosphotransferase, 3′-O-phosphotransferase II (kanamycin, neomycin and G418 resistance). Although the use of antibiotic resistance markers may be most convenient for the transformation of polyploid host cells, preferably however, non-antibiotic resistance markers are used, such as auxotrophic markers (URA3, TRP1, LEU2) or the S. pombe TPI gene (described by Russell P R, 1985, Gene 40: 125-130). Alternatively, a screenable marker such as Green Fluorescent Protein, lacZ, luciferase, chloramphenicol acetyltransferase, or beta-glucuronidase may be incorporated into the nucleic acid constructs of the invention allowing screening for transformed cells.

A variety of selectable marker genes are available for use in the transformation of fungi. Suitable markers include auxotrophic marker genes involved in amino acid or nucleotide metabolism, such as e.g. genes encoding ornithine-transcarbamylases (argB), orotidine-5′-decarboxylases (pyrG, URA3) or glutamine-amido-transferase indoleglycerol-phosphate-synthase phosphoribosyl-anthranilate isomerases (trpC), or involved in carbon or nitrogen metabolism, such as e.g. nitrate reductase (niaD) or facA, and antibiotic resistance markers such as genes providing resistance against phleomycin, bleomycin or neomycin (G418). Preferably, bidirectional selection markers are used for which both a positive and a negative genetic selection is possible. Examples of such bidirectional markers are the pyrG (URA3), facA and amdS genes. Due to their bidirectionality these markers can be deleted from transformed filamentous fungus while leaving the introduced recombinant DNA molecule in place, in order to obtain fungi that do not contain selectable markers. This essence of this MARKER GENE FREE™ transformation technology is disclosed in EP-A-0 635 574, which is herein incorporated by reference. Of these selectable markers the use of dominant and bidirectional selectable markers such as acetamidase genes like the amdS genes of A. nidulans, A. niger and P. chrysogenum is most preferred, the amdS genes of A. niger and P. chrysogenum are disclosed in U.S. Pat. No. 6,548,285. In addition to their bidirectionality these markers provide the advantage that they are dominant selectable markers that, the use of which does not require mutant (auxotrophic) strains, but which can be used directly in wild type strains.

Optional further elements that may be present in the nucleic acid constructs of the invention include, but are not limited to, one or more leader sequences, enhancers, integration factors, and/or reporter genes, intron sequences, centromers, telomers and/or matrix attachment (MAR) sequences. The nucleic acid constructs of the invention may further comprise a sequence for autonomous replication, such as an ARS sequence. Suitable episomal nucleic acid constructs may e.g. be based on the yeast 2μ or pKD1 (Fleer et al., 1991, Biotechnology 9: 968-975) plasmids. An autonomously maintained nucleic acid construct suitable for filamentous fungi may comprise the AMA1-sequence (see e.g. Aleksenko and Clutterbuck (1997), Fungal Genet. Biol. 21: 373-397). Alternatively the nucleic acid construct may comprise sequences for integration, preferably by homologous recombination (see e.g. WO98/46772), or gene replacement (see e.g. EP0 357 127). Such sequences may thus be sequences homologous to the target site for integration in the host cell's genome. In order to promote targeted integration, the cloning vector is preferably linearized prior to transformation of the host cell. Linearization is preferably performed such that at least one but preferably either end of the cloning vector is flanked by sequences homologous to the target locus. The length of the homologous sequences flanking the target locus is preferably at least 30 bp, preferably at least 509 bp, preferably at least 0.1 kb, even preferably at least 0.2 kb, more preferably at least 0.5 kb, even more preferably at least 1 kb, most preferably at least 2 kb. Preferably, the efficiency of targeted integration into the genome of the host cell, i.e. integration in a predetermined target locus, is increased by augmented homologous recombination abilities of the host cell. Such phenotype of the cell preferably involves a deficient ku70 gene as described in WO2005/095624. WO2005/095624 discloses a preferred method to obtain a filamentous fungal cell comprising increased efficiency of targeted integration. Preferably, the DNA sequence in the cloning vector, which is homologous to the target locus is derived from a highly expressed locus meaning that it is derived from a gene, which is capable of high expression level in the filamentous fungal host cell. A gene capable of high expression level, i.e. a highly expressed gene, is herein defined as a gene whose mRNA can make up at least 0.5% (w/w) of the total cellular mRNA, e.g. under induced conditions, or alternatively, a gene whose gene product can make up at least 1% (w/w) of the total cellular protein, or, in case of a secreted gene product, can be secreted to a level of at least 0.1 g/l (as described in EP 357 127 B1). A number of preferred highly expressed fungal genes are given by way of example: the amylase, glucoamylase, alcohol dehydrogenase, xylanase, glyceraldehyde-phosphate dehydrogenase or cellobiohydrolase (cbh) genes from Aspergilli or Trichoderma. Most preferred highly expressed genes for these purposes are a glucoamylase gene, preferably an A. niger glucoamylase gene, an A. oryzae TAKA-amylase gene, an A. nidulans gpdA gene, a Trichoderma reesei cbh gene, preferably cbh1.

More than one copy of a nucleic acid sequence encoding a polypeptide may be inserted into the host cell to increase production of the gene product. This can be done, preferably by integrating into its genome copies of the DNA sequence, more preferably by targeting the integration of the DNA sequence at one of the highly expressed locus defined in the former paragraph. Alternatively, this can be done by including an amplifiable selectable marker gene with the nucleic acid sequence where cells containing amplified copies of the selectable marker gene, and thereby additional copies of the nucleic acid sequence, can be selected for by cultivating the cells in the presence of the appropriate selectable agent. To increase the copy number of the integrated nucleic acid constructs of the invention even more, the technique of gene conversion as described in WO98/46772 may be used.

The nucleic acid constructs of the invention can be provided in a manner known per se, which generally involves techniques such as restricting and linking nucleic acids/nucleic acid sequences, for which reference is made to the standard handbooks, such as Sambrook and Russel (2001) “Molecular Cloning: A Laboratory Manual (3^{rd} edition), Cold Spring Harbor Laboratory, Cold Spring Harbor Laboratory Press, or F. Ausubel et al, eds., “Current protocols in molecular biology”, Green Publishing and Wiley Interscience, New York (1987).

In a further aspect the invention relates to fermentation processes in which the transformed host cells of the invention are used for the conversion of a substrate into the fermentation product A preferred fermentation process is an aerobic fermentation process. The fermentation process may either be a submerged or a solid state fermentation process.

In a solid state fermentation process (sometimes referred to as semi-solid state fermentation) the transformed host cells are fermenting on a solid medium that provides anchorage points for the fungus in the absence of any freely flowing substance. The amount of water in the solid medium can be any amount of water. For example, the solid medium could be almost dry, or it could be slushy. A person skilled in the art knows that the terms “solid state fermentation” and “semi-solid state fermentation” are interchangeable. A wide variety of solid state fermentation devices have previously been described (for review see, Larroche et al., “Special Transformation Processes Using Fungal Spores and Immobilized Cells”, Adv. Biochem. Eng. Biotech., (1997), Vol 55, pp. 179; Roussos et al., “Zymotis: A large Scale Solid State Fermenter”, Applied Biochemistry and Biotechnology, (1993), Vol. 42, pp. 37-52; Smits et al., “Solid-State Fermentation-A Mini Review, 1998), Agro-Food-Industry Hi-Tech, March/April, pp. 29-36). These devices fall within two categories, those categories being static systems and agitated systems. In static systems, the solid media is stationary throughout the fermentation process. Examples of static systems used for solid state fermentation include flasks, petri dishes, trays, fixed bed columns, and ovens. Agitated systems provide a means for mixing the solid media during the fermentation process. One example of an agitated system is a rotating drum (Larroche et al., supra). In a submerged fermentation process on the other hand, the transformed fungal host cells are fermenting while being submerged in a liquid medium, usually in a stirred tank fermenter as are well known in the art, although also other types of fermenters such as e.g. airlift-type fermenters may also be applied (see e.g. U.S. Pat. No. 6,746,862).

In a preferred fermentation process of the invention, one or more fermentation parameters of the process with the transformed host cell is at least 5, 10, 20, 50, 100, 200, or 500% higher than in an otherwise identical process with the untransformed host cell. These fermentation parameters include at least one of: (a) oxygen uptake rate; (b) biomass density; (c) volumetric productivity; and, (d) yield coefficient of fermentation product produced over substrate, whereby these parameters are defined as described herein above and may be determined by methods known in the art.

The fermentation product produced in the fermentation processes of the invention may a primary metabolite, secondary metabolite, a peptide or it may include biomass comprising the host cell itself. The fermentation product may be an organic compound selected from glucaric acid, gluconic acid, glutaric acid, adipic acid, succinic acid, tartaric acid, oxalic acid, acetic acid, lactic acid, formic acid, malic acid, maleic acid, malonic acid, citric acid, fumaric acid, itaconic acid, levulinic acid, xylonic acid, aconitic acid, ascorbic acid, kojic acid, comeric acid, an amino acid, a poly unsaturated fatty acid, ethanol, 1,3-propane-diol, ethylene, glycerol, xylitol, carotene, astaxanthin, lycopene and lutein. Alternatively, the fermentation product may be a β-lactam antibiotic such as Penicillin G or Penicillin V and fermentative derivatives thereof, a cephalosporin, cyclosporin or lovastatin.

In a preferred embodiment of the process the fermentation product is a peptide selected from an oligopeptide, a polypeptide, a (pharmaceutical or industrial) protein and an enzyme. In such processes the peptide is preferably secreted from the host cell, more preferably secreted into the culture medium such that the peptide may easily be recovered by separation of the host cellular biomass and culture medium comprising the peptide, e.g. by centrifugation or (ultra)filtration.

Examples of proteins or (poly)peptides with industrial applications that may be produced in the methods of the invention include enzymes such as e.g. lipases (e.g. used in the detergent industry), proteases (used inter alia in the detergent industry, in brewing and the like), carbohydrases and cell wall degrading enzymes (such as, amylases, glucosidases, cellulases, pectinases, beta-1,3/4- and beta-1,6-glucanases, rhamnoga-lacturonases, mannanases, xylanases, pullulanases, galactanases, esterases and the like, used in fruit processing, wine making and the like or in feed), phytases, phospholipases, glycosidases (such as amylases, beta.-glucosidases, arabinofuranosidases, rhamnosidases, apiosidases and the like), dairy enzymes and products (e.g. chymosin, casein), polypeptides (e.g. poly-lysine and the like, cyanophycin and its derivatives). Mammalian, and preferably human, polypeptides with therapeutic, cosmetic or diagnostic applications include, but are not limited to, collagen and gelatin, insulin, serum albumin (HSA), lactoferrin and immunoglobulins, including fragments thereof. The polypeptide may be an antibody or a part thereof, an antigen, a clotting factor, an enzyme, a hormone or a hormone variant, a receptor or parts thereof, a regulatory protein, a structural protein, a reporter, or a transport protein, protein involved in secretion process, protein involved in folding process, chaperone, peptide amino acid transporter, glycosylation factor, transcription factor, synthetic peptide or oligopeptide, intracellular protein. The intracellular protein may be an enzyme such as, a protease, ceramidases, epoxide hydrolase, aminopeptidase, acylases, aldolase, hydroxylase, aminopeptidase, lipase.

In another aspect the invention relates to a nucleic acid molecule comprising a nucleotide sequence that encodes a fungal oxygen-binding protein. The nucleotide sequence is preferably selected from: (a) nucleotide sequences encoding a polypeptide comprising an amino acid sequence that has at least 66, 67, 68, 70, 75, 80, 85, 90, 95, or 98% sequence identity with the amino acid sequence of SEQ ID NO. 3; (b) nucleotide sequences the complementary strand of which hybridizes to a nucleotide sequence of (a); and, (c) nucleotide sequences the sequence of which differs from the sequence of a nucleotide sequence of (b) due to the degeneracy of the genetic code. The fungal oxygen binding protein preferably is a flavohemoglobin as defined above. Preferably the flavohemoglobin is from an Aspergillus, more preferably from A. niger or a related species as defined in the definitions section. An example a nucleotide sequence encoding an Aspergillus flavohemoglobin is provided in SEQ ID NO. 4. Preferred nucleotide sequences are at least 50, 60, 70, 80 or 90% identical to SEQ ID NO. 4, or hybridise to SEQ ID NO. 4 under moderate, preferably under stringent conditions.

Another preferred nucleic acid molecule comprises a nucleotide sequence that encoding a fungal oxygen-binding protein wherein the nucleotide sequence is preferably selected from: (a) nucleotide sequences encoding a polypeptide comprising an amino acid sequence that has at least 78, 79, 80, 85, 90, 95, 98, or 100% sequence identity with the amino acid sequence of SEQ ID NO. 2; (b) nucleotide sequences the complementary strand of which hybridises to a nucleotide sequence of (a); and, (c) nucleotide sequences the sequence of which differs from the sequence of a nucleotide sequence of (b) due to the degeneracy of the genetic code. The fungal oxygen-binding protein preferably is a hemoglobin domain from a fungal flavohemoglobin as defined above. Preferably the hemoglobin domain is from an Aspergillus, more preferably from A. niger or a related black Aspergillus.

Yet another preferred nucleic acid molecule comprises a nucleotide sequence that encoding a fungal oxygen-binding protein wherein the nucleotide sequence is preferably selected from: (a) nucleotide sequences encoding a polypeptide comprising an amino acid sequence that has at least 83, 84, 85, 90, 95, or 98% sequence identity with the amino acid sequence of SEQ ID NO. 1; (b) nucleotide sequences the complementary strand of which hybridises to a nucleotide sequence sequence of (a); and, (c) nucleotide sequences the sequence of which differs from the sequence of a nucleotide sequence of (b) due to the degeneracy of the genetic code. The fungal oxygen-binding protein preferably is a hemoglobin domain from a fungal flavohemoglobin as defined above. Preferably the hemoglobin domain is from an Aspergillus, more preferably from A. oryzae or another species from the Aspergillus section Flavi (e.g. A. sojae). An example a nucleotide sequence encoding an A. oryzae hemoglobin domain is provided in SEQ ID NO. 5. Preferred nucleotide sequences are at least 50, 60, 70, 80 or 90% identical to SEQ ID NO. 5, or hybridise to SEQ ID NO. 5 under moderate, preferably under stringent conditions.

Preferred nucleic acid molecules or nucleotide sequences according to the invention are isolated nucleic acid molecules or nucleotide sequences. Preferably, the nucleic acid molecules according to the invention, when present in an expression construct upon transformation of a fungal host cell with the construct, confers to the host cell an increase in a fermentation parameter compared to an otherwise identical host cell that is not transformed with the construct, whereby the fermentation parameter is at least one of: (a) oxygen uptake rate; (b) biomass density; (c) volumetric productivity; and, (d) yield coefficient of fermentation product produced over substrate; whereby the fermentation parameters are defined and/or determines as described above. Preferably, at least one fermentation parameter of the transformed host cell is increased by at least 5, 10, 20, 50, 100, 200, or 500% as compared to the untransformed host cell.

In a further aspect the invention pertains to a polypeptide comprising an amino acid sequence selected from: (a) amino acids sequences that have at least 66, 67, 68, 70, 75, 80, 85, 90, 95, or 98% sequence identity with the amino acid sequence of SEQ ID NO. 3; (b) amino acids sequences that have at least 78, 79, 80, 85, 90, 95, 98, or 100% sequence identity with the amino acid sequence of SEQ ID NO. 2; and, (c) amino acids sequences that have at least 83, 84, 85, 90, 95, or 98% sequence identity with the amino acid sequence of SEQ ID NO. 1. Preferably the polypeptide is an isolated polypeptide. A preferred polypeptide is a peptide that when expressed in a fungal host cell from an expression construct comprising a nucleotide sequence encoding the polypeptide, upon transformation of the host cell with the expression construct, confers to the host cell an increase in a fermentation parameter compared to an otherwise identical host cell that is not transformed with the construct, whereby the fermentation parameter is at least one of: (a) oxygen uptake rate; (b) biomass density; (c) volumetric productivity; and, (d) yield coefficient of fermentation product produced over substrate; whereby the fermentation parameters are defined and/or determines as described above. Preferably, at least one fermentation parameter of the transformed host cell is increased by at least 5, 10, 20, 50, 100, 200, or 500% as compared to the untransformed host cell.

### DESCRIPTION OF THE FIGURES

FIG. 1. Phylogenetic analysis of flavoHb proteins from fungal species, A. eutrophus and E. coli. The tree was constructed using neighbor-joining method from ClustalW1.82 (Saitou and Nei 1987). Abbreviations are as shown in Table 1.

FIG. 2. Amino acid alignment of N-terminal truncated (**) flavoHb proteins of A. niger and the N-terminal truncated (**) flavoHb and non-truncated flavoHb proteins of the Pezizomycotina to the A. eutropus (Ermler et al., 1995) flavoHb protein. The abbreviations are as shown in Table 1. Bold-faced residues marked with an asterisk represent important residues (see text for details). The 6 α-helices of the hemoglobin domain are marked (A, B, C, E, F, G, H) as well as the different secondary structures in the FAD binding domain (Fα/β) and the NADP(H) domain (Nα/β in between > <.

FIG. 3. Transcript levels of the fhbA gene during growth of A. oryzae in 2% WLM (lane 1: 17 hrs, 2: 24 hrs, 3 42 hrs, 4: 53 hrs), 2% WSM (lane 5: 2 days, 6: 3 days, 7: 4 days), wheat kernels (lane 8: 3 days, 9: 4 days, 10: 5 days). Alternatively A. oryzae was grown for 53 hours in 2% WLM and transferred to 2% WLM for either 0 (lane 11), 2 (lane 12), 4 (lane 13), 6 (lane 14), 8 (lane 15) or 30 (lane 16) hours. The Biomass (weight (g)) and Glucose concentration (Glu (g/L)) in the growth medium or the extracts of the growth medium were determined as described below.

FIG. 4. Transcription of the fhbA gene during polarized growth of A. oryzae. Northern analysis after transfer of 72 hrs grown A. oryzae in 2% WSM to fresh 2% WSM for 6 (lane 1) or 9 hours (lane 2) or transfer to agar medium (WAM) for 6 (lane 3) or 9 hours (lane 4). Transcriptional analysis during growth of A. oryzae in 2% WLM without shaking after 48 (lane 5), 72 (lane 6), 96 (lane 7) and 120 (lane 8) hrs. The drawings represent a schematic progress of fungal polarized growth. The horizontal line in the drawing represents the 2% WSM/air interface. The wild-type (lane 9) and pclA disrupted (lane 10) strains were grown for 72 hours and transferred for 6 hours to fresh 2% WSM. MpkA represents transcriptional analysis with the probe for the mpkA gene. Moreover, the wildtype (lane 11) and pclA disrupted (lane 12) A. oryzae strains were grown for 3 days on wheat kernels and fhbA gene transcription was analyzed.

FIG. 5 A. Schematic representation of the A. niger and the partial A. oryzae flavohemoglobin protein, and the Vitreoscilla hemoglobin domain. The hemoglobin domains are shown as white boxes. The black box at the N-terminus (N) represents the N-terminal extension of the A. niger protein. The black box at the C-terminal side (C) of the A. niger protein represents the reductase domain. The unknown part of the A. oryzae reductase domain is shown as a dotted line.

FIG. 5 B. Alignment of the predicted amino acid sequence of the A. niger (AN; CAF32308.1), A. oryzae (AO; CAF32307.1), and Vitreoscilla (VT; P04252) hemoglobin domain sequences using clustalW 1.82. > <mark the limits of the domains. Identical amino acids are shown in bold. The secondary α-helixes (A, B, E, F, G, H) and the residues that might be involved in hemoglobin functionality (B10: Y, CD1: F, E7: Q, E11: L, F7: K, H8: H, G5: Y, H23: E) are marked with an asterisk. The amino acids were identified according to Frey and Kalio (2003), see text for further details.

FIG. 6. Dissolved oxygen (DO (%)) measured in oxygen saturated complete medium (CM) after addition of 3 g wet weight wild-type (closed triangles) and hemoglobin-producing strains (pHBN: closed squares, pHBO: open circles) transformants. The open triangles represent time course with 15 g of wild-type cells, and the open squares represent that of CM without addition of biomass. Results are the average of 2 independent experiments.

FIG. 7. Biomass development expressed as gram wet weight of the wild-type (close triangle) and hemoglobin-producing strains (pHBN: close squares, pHBO: open circles) during cultivation for 120 hours in minimal medium (A), 5% WSM (B) and PDA (C).

### EXAMPLES

### 1. Example 1

### Isolation of the fhbA Gene of A. niger and Expression of the A. oryzae fhbA Gene During Polarized Growth

### 1.1 Materials and Methods

### 1.1.1 Strains and Media

A. oryzae ATCC16168 was used throughout this study and A. niger CBS120.49 was used to isolate the flavohemoglobin encoding gene. The pclA disrupted A. oryzae strains were constructed as described (WO 01/09352). Growth on wheat kernels, in 2% wheat based liquid medium (2% WLM) and growth on 2% wheat based solid medium (2% WSM) was performed as described (te Biesebeke et al., 2002; 2004). The transfer of fungal biomass to 2% WLM, 2% WSM or water agar medium (WAM) was performed as described (te Biesebeke et al., 2004). WAM was prepared by weighing 2 g bacterial agar (Difco) in 100 ml H₂O that was sterilized by heating for 15 min to 120° C. and poured in sterile petri dishes. For surface growth on 2% WLM, 10⁶ A. oryzae conidia/ml were inoculated in a 250 ml shake flask containing 100 ml 2% WLM and incubated without shaking at 30° C.

1.1.2 Isolation of the fhbA Gene of A. niger

In an heterologous macroarray analysis similar as described in te Biesebeke et al., (2005) a cDNA clone was identified that showed differential hybridization intensity with labeled first strand cDNA of total RNA from A. oryzae grown in 2% WSM compared to that grown on wheat kernels. The complete cDNA was PCR amplified with primers MBL1588 and MBL1589 (te Biesebeke et al., 2005) using 40 cycles of 30s at 94° C., 1 min at 45° C., 30s at 72° C. The DNA fragment was purified from 1% agarose gel electrophoresis with the Qiaquick DNAeasy columns (Qiagen, UK) and cloned in pGEM-T easy vectors (PROMEGA) and sequenced. Sequencing was performed with the Cycle Sequencing Kit from Pharmacia according to the manufacturer protocol. Sequence data were obtained with the ABI Prism 310 Genetic Analyzer from Applied Biosystems (Perkin-Elmer division). The complete cDNA was isolated from the A. niger cDNA library (Veldhuisen et al., 1997), sequenced and the cDNA sequence and the deduced amino acid sequence was deposited at the EMBL database under the respective numbers AJ627189 and CAF25490.1.

1.1.3 Blast Searches, Homology. ClustalW and Phylogentic Tree Construction

The cDNA sequence was matched to different databases as described (te Biesebeke et al., 2005) in blast searches (Altschul et al., 1997) to obtain homologous sequences of other fungi. Homology between Aspergillus DNA sequences was determined with blast 2 sequences (Tatusova and Madden 1999). Homologous protein sequences were submitted for ClustalW 1.82 (Thompson et al., 1994) alignment at FMBL-EBI (www.ebi.ac.uk). The phylogenetic tree of Ascomycota and Basidiomycota flavohemoglobins was constructed using the neighbor-joining method (Saitou and Nei 1987) from ClustalW 1.82.

### 1.1.4 Southern and Northern Analysis

Southern and Northern analysis was performed as described (te Biesebeke et al., 2004). Northern analysis was performed with ³²P labeled (Random Prime Labeling Kit, Pharmacia) A. niger probes for the MapkA and flavohemoglobin genes. The probe for MapkA (db. Acc. Nr. AY540623) was amplified from the pGEM-T vector containing the DNA fragment from MapkA that was a kind gift from Dr. Arthur Ram from Leiden University. The A. niger mpkA sequence (db. acc. nr. AY540623) has high homology (203 of 254 identical nucleotides) to the A. oryzae mpkA gene (db. acc. nr. BAD12561) determined by blast 2 sequences (Tatusova and Madden 1999) allowing specific hybridization under the chosen conditions (Howley et al., 1979). The probe for flavohemoglobin was PCR amplified from the above mentioned pGEM-T vector containing the flavoHb cDNA sequence (Db. Acc. Nr. AJ627189). Probes were purified from 1% agarose gel with Qiaquick DNAeasy columns (Qiagen, UK).

### 1.2 Results

1.2.1 Isolation of a Putative fhbA Encoding Gene from A. niger

In a previous study (te Biesebeke et al., 2005) a heterologous macroarray analysis was used to identified genes associated with the growth phenotype of A. oryzae grown on wheat kernels and in 2% WLM. From this type of analysis a cDNA clone was identified showing differential hybridization with probes for total RNA from A. oryzae grown in 2% WSM compared to that grown on wheat kernels. The complete cDNA of this clone was sequenced (Db. Acc. Nr. CAF254990.01) and its deduced amino acid sequence was identified as a protein homologous to the flavohemoglobin (flavoHb) of Alcaligenes eutrophus (Ermler et al., 1995). Based on the cDNA sequence primers were designed to PCR amplify and sequence the genomic copy of the A. niger flavoHb gene (fhbA) Based on the sequence of the PCR fragment, the A. niger fhbA gene did not contain any introns.

### 1.2.2 Phylogenetic Analysis of Fungal Flavohemoglobins

Comparison of the A. niger flavoHb protein sequence with several publicly available fungal sequence databases revealed a number of related flavohemoglobin sequences. Remarkably, several fungal species of which the full genomes are available in public databases (Aspergillus nidulans, Neurospora crassa, Gibberella zea (Fusarium graminearum), Debaryomyces hansenii (Candida famata) and Podospora anserina have 2 genes encoding putative flavoHb proteins in their genome (Table 1). Candida albicans has 3 flavoHb genes (Ullman et al 2004), whereas Aspergillus fumigatus, Magnaporthe grisae, Phanerochaete chrysosporium, Crypotococcus neoformans, S. cerevisiae and S. pombe have only a single flavoHb encoding gene in their genomes (Table 1).

Interestingly, the overall sequence identity of the A. niger FlavoHb protein compared to most other fungal or yeast flavoHb sequences but also to the A. eutropus and E. coli flacoHb sequences is in the range of 30-45%, with exception of the A. fumigatus and A. nidulans sequences (Table 1). A clear different feature of the A. niger flavoHb compared to that of most other fungal flavoHb proteins is the N-terminal extension with 43 amino acid residues. Only P. chrysosporium, M. grisae, C. neoformans and S. pombe have N-terminal extensions of respectively 15, 24, 79 and 83 amino acid residues.

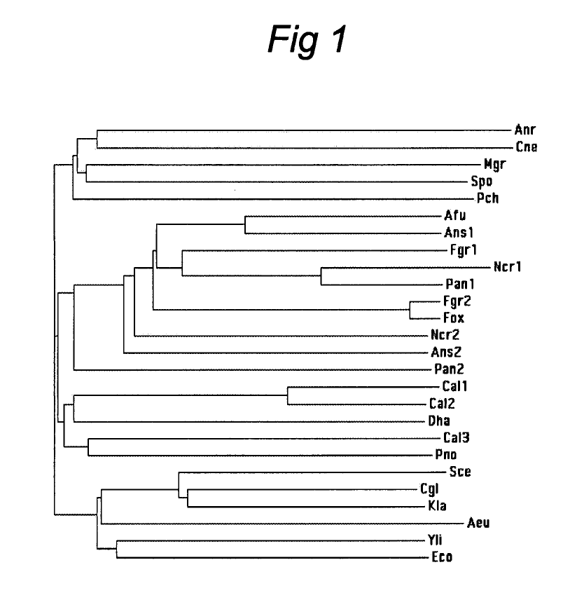

Phylogenetic analysis of the fungal flavohemoglobin protein sequences of Table 1 shows that the flavoHb proteins with N-terminal extensions, including the Basidiomycota C. neoformans and P. chrysosporium, cluster together in a separate group (FIG. 1). The Pezizomycotina flavoHb proteins from Aspergillus, Neurospora, Podospora and Fusarium species form a distinct group compared to the other Saccharomycotina flavohemoglobins from Saccharomyces, Pichia, Kluyveromyces, Yarrowia, Candida and Debaryomyces species (FIG. 1). The bacterial flavoHb proteins of A. eutrophus and E. coli group together with the Saccharomycotina flavoHb proteins. Another interesting observation is that the different flavoHb's from the same species do not cluster close to each other in the phylogram, with exception of Cal1 and Cal2. In general, the results as presented in FIG. 1 show that the different fungal and yeast flavoHb proteins are unusually divergent in sequence.

1.2.3 Conserved Amino Acids of Filamentous Fungal flavoHb

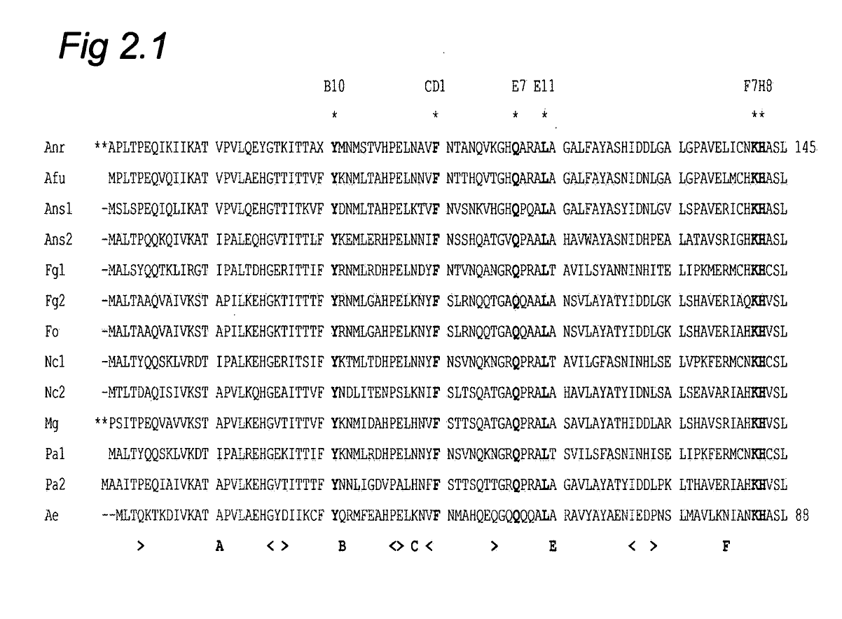

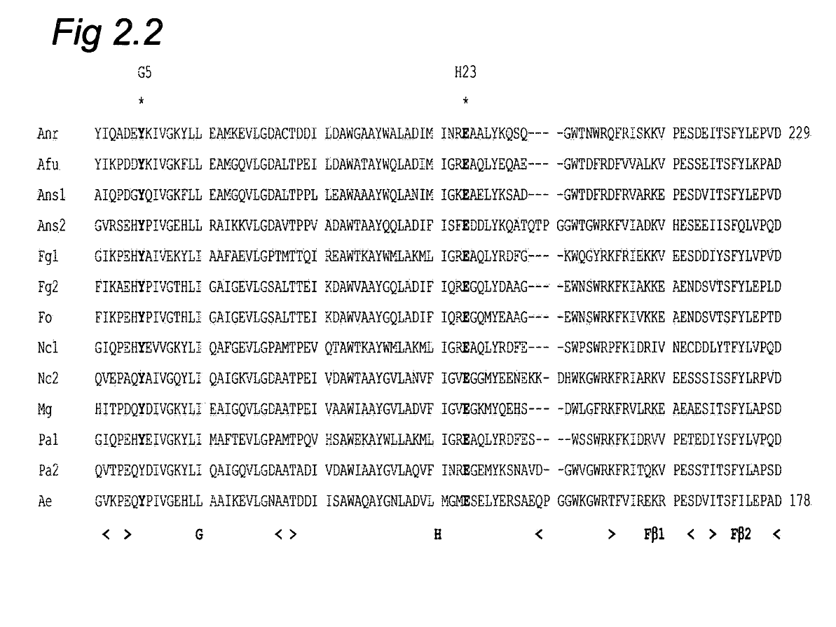

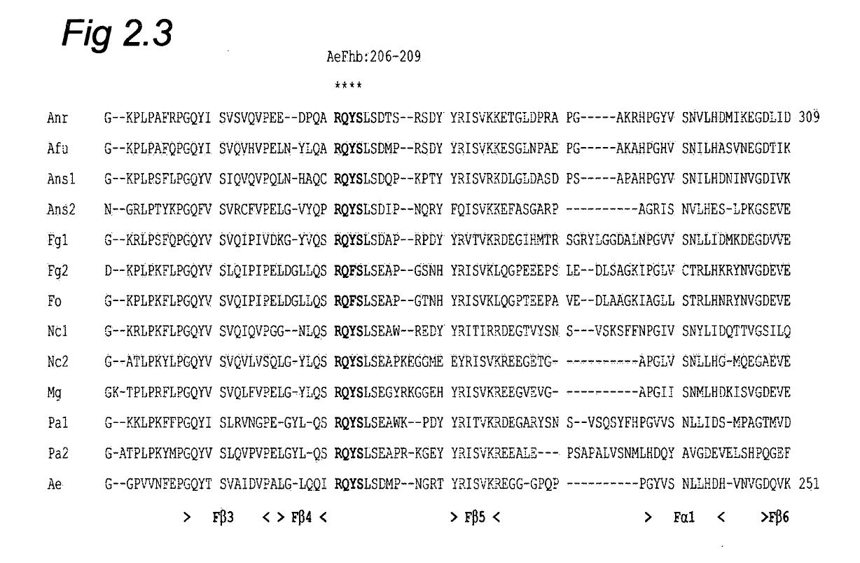

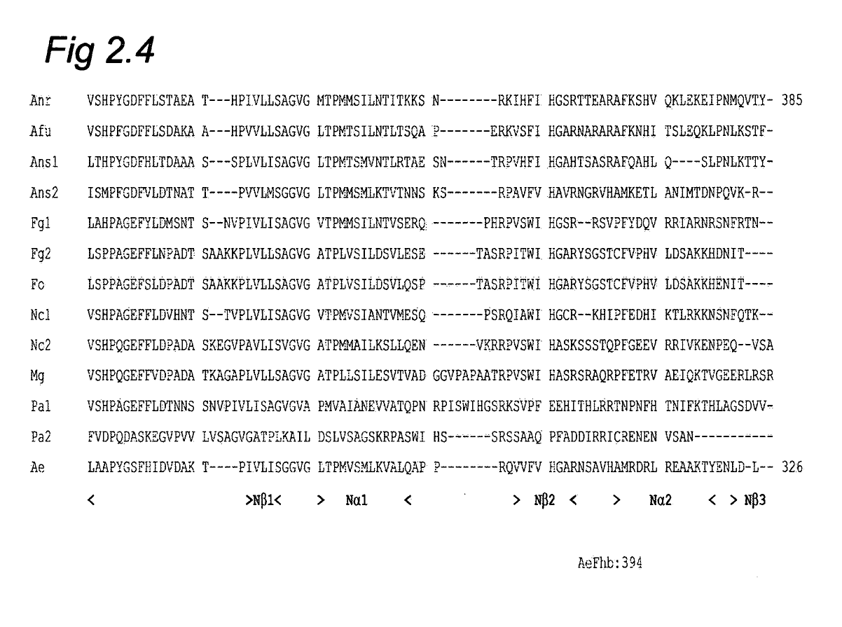

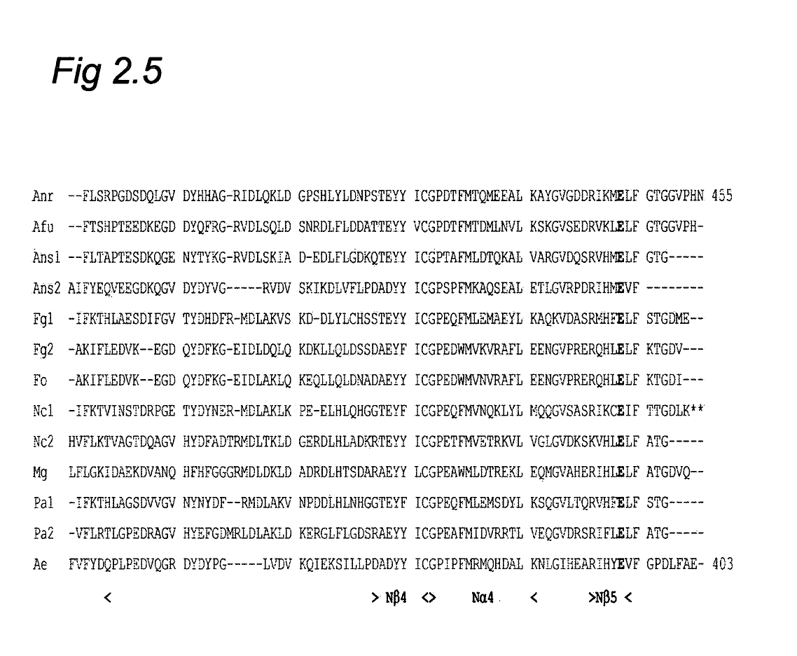

Table 1 and FIG. 1 suggest low sequence identity between the putative filamentous fungal flavoHb proteins. To determine whether the different filamentous fungal flavoHb sequences share homology in functionally relevant residues, the amino acid sequences were aligned to the FlavoHb sequence of A. eutrophus of which the three-dimensional structures has been elucidated and functional relevant residues have been determined (Elmer et al., 1995). The flavoHb of A. eutrophus is made up of a hemoglobin, FAD and NAD binding domain (Ermler et al., 1995, Ilari et al., 2002) (FIG. 2). The globin domain ranging from residue 1 to 147 (A. eutropus), consists of 6 α-helices (A, B, C, E, F, G, H) and holds the heme molecule that is embedded in a hydrophobic crevice formed by 6 alpha helices (Weber and Vinogradov 2001, Ilari et al., 2002, Frey and Kallio 2003). A number of residues in the globin domains are invariant according to all known flavoHb protein sequences. The Tyr-B10 and Gln-E7 have been suggested to be involved in stabilization of the heme bound dioxygen (Frey and Kallio 2003) and are conserved in the globin domain of the filamentous fungal flavoHb proteins. His-F8 in α-helix F, Tyr-G5 in helix G and Glu-H23 in helix H are suggested to form the catalytic triad at the proximal site by modulating redox properties of the heme-iron atom (Frey and Kallio 2003) and are conserved in the globin domain of the filamentous fungal flavoHb proteins. The FAD and NAD binding domain ranges from the respective residues 153 to 266 and residue 267 to 397 in the A. eutrophus sequence (Ermler et al., 1995). The FAD binding domain consists of a six-stranded antiparallel β-barrel (Fβ1-6) capped by a helix (Fα1) (Erlmer et al., 1995). The residues 206-209 (A. eutrophus) in the loop between sheet Fβ4 and Fβ5 are involved in FAD binding (Frey and Kallio 2003) and are conserved in the suggested FAD domain of the filamentous fungal flavoHb proteins. The NAD binding domain is built up of a five-stranded parallel β-sheet flanked by 2 helices (Nα1, Nα2) on one side and by one helix (Nα4) at the other side (Erlmer et al., 1995). The conserved Lys-F7 in α-helix F and Glu-394 in sheet Nβ5 are amongst other residues, considered to be essential for transport of electrons from FAD to the heme iron (Frey and Kallio 2003).

1.2.4 A. oryzae fhbA Gene transcription

An A. oryzae flavoHb protein-encoding gene is unknown and different approaches to isolate the full-length gene sequence were unsuccessful. Therefore we decided to use a PCR amplified probe from the A. niger fhbA gene to study transcriptional regulation of the A. oryzae fhbA gene. Sequence similarity between these two species suggests specific hybridisation under the chosen conditions (te Biesebeke et al., 2005). Moreover, heterologous Southern analysis with the A. niger fhbA probe and chromosomal DNA of A. oryzae revealed a single hybridizing band showing that this probe is specific for a single copy FlavoHb gene from A. oryzae. Therefor, the A. niger fhbA probe was used to detect transcript levels of the A. oryzae fhbA gene.

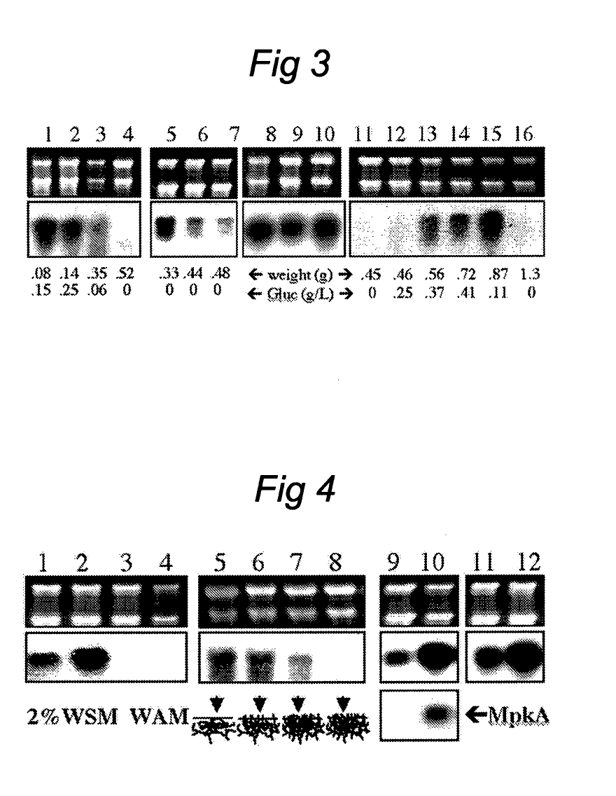

To determine the growth conditions under which transcription of the fhbA gene occurs, A. oryzae was grown in 2% WLM, on 2% WSM and on wheat kernels. In Northern analysis it is shown that the A. oryzae fhbA gene transcript level is highest in the “logaritmic” growth phase in 2% WLM at 17 and 24 hours (FIG. 3, lane 1 and 2) and on 2% WSM after 48 hrs of growth (FIG. 3, lane 5). The correlation between the “logaritmic” growth phase and fhbA gene transcription was further corroborated in Northern analysis with total RNA of A. oryzae grown for 3, 4 and 5 days on wheat kernels (FIG. 3, lanes 8-10). Although a continuous increase in biomass can not be determined accurately under these cultivation conditions, the fact that oxygen uptake rate is still increasing during growth of A. oryzae on wheat kernels (Rahardjo et al., 2001) confirms continuing growth.

The results in FIG. 3 (lanes 1-10) are in agreement with the results shown for S. cerevisiae that YHB1 is expressed during logaritmic growth (Crawford et al., 1995). These results suggest that besides regulation by the heme-activated protein, the transcription of the YHB1 gene might be dependent on polarized growth or the amount of biomass. To analyze if the amount of A. oryzae biomass affects fhbA transcript levels, cultures were grown for 53 hours until the maximum amount of biomass was produced in 25 ml 2% WLM. Subsequently, biomass was harvested after filtration through miracloth and transferred to 25 ml 2% WLM and the fhbA gene transcription was analyzed. The transcript levels of the A. oryzae fhbA gene re-appeared after 4, 6 and 8 hours transfer (FIG. 3 lane 11-16) and was disappeared again after 30 hours. These results show that the absence of transcript at 53 hrs is not the effect of the amount of biomass produced because increase in biomass formation after transfer to fresh medium resulted in renewed fhbA gene transcription. This suggests that fhbA gene transcription is induced by polarized growth or repressed by starvation.

1.2.5 fhbA Gene Transcription Appears During Polarized Growth

Two other experimental approaches were performed to study the correlation between polarized growth, starvation and fhbA gene transcription. A. oryzae grown for 4 days on 2% WSM on a membrane was transferred to fresh 2% WSM and to an agar plate with only water (WAM). There was no difference in biomass observed after 6 and 9 hrs transfer to either 2% WSM and WAM. However, newly formed penetrative hyphae were observed and transcript levels of the fhbA gene were detected only in 2% WSM after 6 and 9 hours transfer (FIG. 4, lane 1-4). In another approach, shake flasks with 2% WLM were inoculated and incubated without shaking. Compared to 48 hrs of growth, at 72 hours biomass increased and formation of aerial hyphae was observed (See schematic drawing FIG. 4). At 120 hrs no biomass and macroscopic difference was observed compared to that at 96 hrs. FIG. 3 shows that transcript levels of the A. oryzae fhbA gene were detected during submerged biomass formation (FIG. 4 lane 1), surface growth and aerial hyphae formation (FIG. 4, lane 2) on 2% WLM and disappeared when cells entered stationary growth phase (FIG. 4, lane 4). As suggested before for S. cerevisiae (Gasch et al., 2000) and C. albicans (Nantel et al., 2002) these results (FIG. 3 and FIG. 4, lane 1-4 and 5-8) sustain our suggested relation between flavohemoglobin expression and polarized growth or starvation.

1.2.6 fhbA Gene Transcription in a Strain with Disordered Polarized Growth

Disruption of the pclA (kexB) gene in A. oryzae results in a disordered polarized growth phenotype (Mizutani et al., 2004) resulted in higher transcript levels for the mpkA gene and constitutive increased levels of phosphorylated MpkAp compared to the wildtype (Mizutani et al., 2004). To correlate polarized growth to fhbA gene transcription Northern analysis was performed with total RNA isolated from the wild-type and pclA disrupted strain after 6 hours of membrane transfer assay performed as described (te Biesebeke et al., 2004). FIG. 4 (lane 9-10) showed that the pclA disrupted strain has high transcript levels of the mpkA gene and 5 times higher transcript levels of the fhbA gene compared to the wild type. The wild-type and pclA disrupted strains were also grown on wheat kernels for 3 days. Northern analysis with total RNA isolated from the wild-type and pclA disrupted strain revealed that on the wheat kernels the expression of the fhbA gene was about 2 times higher compared to the wild type (FIG. 4, lane 11-12).

### 2. Example 2

### Overproduction of Aspergillus Hemoglobin Domains in Aspergillus

### 2.1 Materials and Methods

2.1.1 Strains and media

A. oryzae ATCC16168 was used throughout this study. Growth on ground wheat kernels and 5% wheat based solid medium (5% WSM) was performed as described (te Biesebeke et al., 2002; 2004). Potato dextrose agar (Oxoid) (PDA) was prepared as described by the manufacturer. Complete medium (CM) consisted of 1% glucose, 0.1% Yeast extract, 0.1% casamino-acids, 0.2% peptone, 2 mM MgSO₄, 10 mM NaNO₃, spore elements. Minimal medium is CM without peptone, yeast extract and casamino-acids. For membrane cultures Nitrocellulose membranes (3 μm pore size, Millipore) that were placed on 25 ml of the agar-solidified substrates in petridishes innoculated with 2.5×10⁷ spores as described (te Biesebeke et al., 2004).

### 2.1.2 Isolation of the Hemoglobin Domain Encoding DNA Fragments.

To amplify the DNA fragment (444 nucleotides) of the hemoglobin gene of A. niger, primers 57ANFHB1 (5′CATGCCATGGCGCTCACACCAGAGCAGATC3′) and 58ANHB2 (5′GGAAGATCTTTAGCCCTGGCTTTGCTTGTAGAGTGC3′) were designed on the basis of the flavohemoglobin encoding gene (AJ629189). To amplify the DNA fragment (444 nucleotides) of the hemoglobin domain of A. oryzae, primers 50HbAONCO (5 ′CATGCCATGGCGCTCTCCCCTGAACAAATC3′) and 53HbOBAM (5′CGCGGATCCTTATCCGTCGGCCTGCTT3′) were designed on the basis of the flavohemoglobin gene from A. nidulans (Acc. Nr. AACD0100122, region: 103592 to 104824) and the AoEST04885 sequence (nrib.go.jp/ken/EST/db/blast.html). Primers were constructed in such a way that NcoI and BamHI restriction sites were introduced in the DNA fragment at the 5′ and 3′ terminal sites respectively. In both 3′-end located primers, a stop codon was introduced at the 5′ site of the BamHI restriction site. Taq DNA polymerase (Boehringer) was used with Aspergillus species chromosomal DNA in 40 cycles PCR amplification (30s at 94° C., 1 min at 45° C., 30s at 72° C.) according to the manufacturer's protocol. The sequence of the DNA fragment of A. oryzae contained a NcoI restriction site that restrained the chosen cloning strategy. Therefore, a silent point mutation was introduced at the NcoI restriction site by using the overlap PCR extension method (Yolon and Shabarova 1990, Yon and Fried 1989) and primers 51HbOmut1 (5′GGACCTCGCCCATTGCCTCCAAC3′) and 52HbOmut2 (5′GTTGGAGGCAATGGGCGAGGTCC3′). Subsequently, the mutated A. oryzae DNA fragment was cloned and sequenced confirming the presence of only the silent mutation. DNA fragments were purified from 1% agarose gel electrophoresis with the Qiaquick DNAeasy columns (Qiagen, UK) and cloned in pGEM-T easy vectors (PROMEGA) and sequenced. Sequencing was performed with the Cycle Sequencing Kit from Pharmacia according to the manufacturer protocol. Sequence data were obtained with the ABI Prism 310 Genetic Analyzer from Applied Biosystems (Perkin-Elmer division). The M13 Forward and Reverse sequencing primers (Table 2) were used for sequence analysis of the cloned hemoglobin DNA fragment from A. niger and A. oryzae. Nucleotide sequences for the A. oryzae and A. niger hemoglobin DNA fragments were assigned Genbank accession numbers: AJ628839 and AJ62840.

### 2.1.3 Construction of the Expression Vectors and Fungal Transformation

Plasmid pAN52-1 Not (Gene bank accession number Z32524) containing the promoter region of the gpdA gene of Aspergillus nidulans was used for all contructs. Plasmid pHBN and pHBO were constructed by introducing the 441 bp NcoI/BamHI digested PCR amplified A. niger and A. oryzae hemoglobin encoding DNA fragment in plasmid pAN52-1 Not. Sequencing of the constructed plasmids confirmed the absence of irregularities.

Plasmids pHBN and pHBO were used in a co-transformation procedure with plasmid pAB4-1 (van Hartingsveldt et al., 1987) containing the Aspergillus niger pyrG selection marker contained the pyrG auxotrophic selection marker gene as described by van den Hondel (1992) to transform the Aspergillus oryzae (ATCC16868) pyrG (te Biesebeke et al., 2002; 2004). Cotransformants were selected for growth in the absence of uridine (Verdoes et al., 1993). From each transformation a dozen of transformants were analyzed with a colony hybridization (Sambrook et al., 2001, supra) performed with a ³²P labeled trpC terminator probe, a DNA fragment which is part of the expression vector pAN52-1Not (db. acc. nr. Z32524) and a single transformation with a positive hybridisation was selected and used for further analysis.

### 2.1.4 Analysis of Hemoglobin Production

Hemoglobin or other oxygen-binding proteins may be assayed as follows. A method to detect the presence of an active hemoglobin was based upon consumption of oxygen of exponentially grown wild-type and transformed cells in complete medium (CM) similar as was described previously (Yu et al., 2002). The quantitative determination of dissolved oxygen (DO) was determined in a shake flask using an oxygen electrode connected with the control system of a New Brunswick fermentor (Yu et al., 2002). Oxygen calibration was carried out with cell free CM medium saturated with oxygen after 15 min of bubbling of pure oxygen through the medium set at 100% saturation. Equal amounts (3 g) of exponentially grown wild-type or transformed cells were transferred to 100 ml 100% oxygen-saturated CM medium and DO changes were measured. As control experiments DO changes were measured in 100 ml CM with 100% oxygen saturation and in 100 ml CM with 100% oxygen-saturation with 15 g of wet weight wild-type biomass. Alternative methods include differential CO spectrum (Webster and Liu, 1974) and the “gassing out” method (Bhave and Chattoo, 2003). Wet biomass weight may be determined after separation of the biomass from the culture medium by filtration (e.g. through Miracloth) or centrifugation.

### 2.1.5 Analysis of Secreted Enzyme Production

Extracts from the wild-type and the A. oryzae transformants harboring pHBN and pHBO were grown for 5 and 6 days on ground wheat kernel or for 3 days on 5% WSM were prepared and analysed for α-amylase, glucoamylase and protease activities as described by te Biesebeke et al (2004).

The protease activity may be measured according to a modified procedure as described by Holm (1980). As a substrate N,N-dimethylcaseine (Sigma, C 9801) was used. 2 μl sample+13 μl water was mixed with 75 μl reagent (5 g/l N,N-dimethylcaseine in 0.1 M K₂HPO₄ (pH=7.0)) and incubated at 37° C. for 17.5 minutes. The reaction was stopped by addition of 185 μl M Na₂B₄O₇.10H₂O/4 mM Na₂SO₃ (pH=9.3) and 5 μl starter 2.5% TNBS (2,4,6,-Trinitrobenzene Sulfonic Acid, Pierce #28997). The absorption at 405 nm was measured after 200 seconds. A glycine delution range was used as a standard. Samples were also incubated in triplo with water to measure the background and the data were corrected for the mean value. The procedure was fully automated using a Cobas Mira Plus Autoanalyser (Roche). One unit of protease activity was defined as the amount of enzyme needed to produce one μmol of amino acids per minute at 37° C. at the indicated pH.

The alpha-amylase activity was determined in the extracts according to the Megazyme (Wicklow, Ireland) alpha-amylase assay procedure (Ceralpha method with ICC standard No. 303) using non-reducing-end blocked p-nitrophenyl maltoheptaoside (BPNPG7) as a substrate to avoid hydrolysis by exo-enzymes such as beta-amylase, amyloglucosidase and alpha-glucosidase. One unit of alpha-amylase activity is defined as the amount of enzyme needed to liberate one μmol of p-nitrophenol per minute at 37° C. at pH 5.5.

The glucoamylase activity was determined using p-Nitrophenyl-maltoside (Megazyme, Wicklow Ireland) according to the manufacturers amyloglucosidase assay (RAMGR3 11/99). One unit of glucoamylase activity is defined as the amount of enzyme needed to produce one μmol of p-nitrophenol per minute at 37° C. at pH 4.5.

Samples used for determination of glucose and amino acid concentrations are boiled for 5 minutes at 95° C. and left at RT until use. Glucose is analyzed enzymatically using the glucose HK 125 method (cat. no. A11A0016) from ABX Diagnostics (Burrin 1985). Amino acids are analyzed using the TNBS method (trinitrobenzenesulfonic acid) described by Adler-Nissen (Adler-Nissen 1979).

### 2.2 Results

### 2.2.1 Cloning of the Aspergillus Hemoglobin-Domain Genes

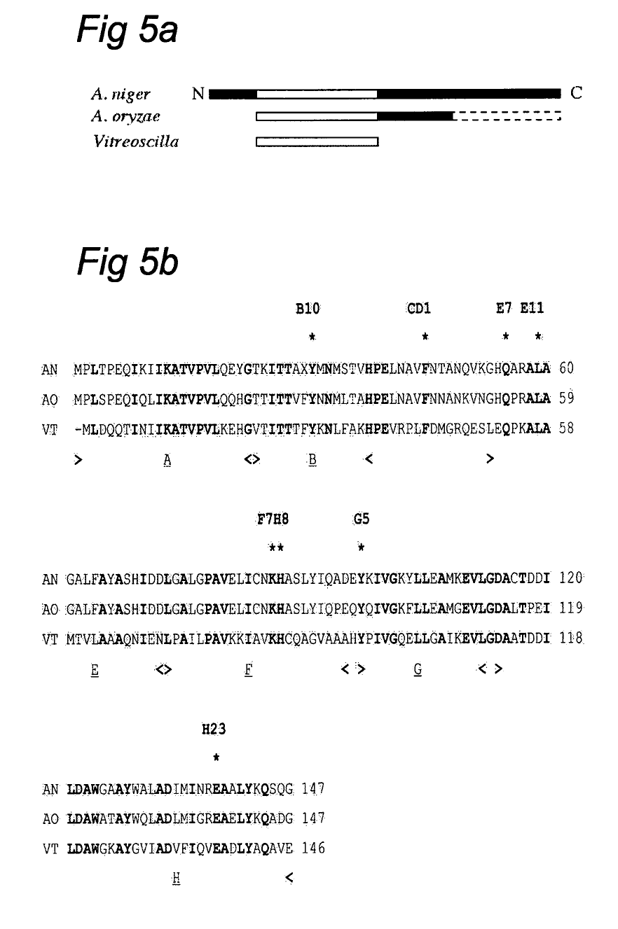

The DNA fragments of the hemoglobin-encoding gene of A. oryzae and A. niger were PCR amplified and subsequently sequenced. The deduced amino acid sequences of the DNA fragments of the A. oryzae and A. niger hemoglobin-domain genes were aligned to that of the Vitreoscilla hemoglobin and the secondary structure elements were assigned (FIG. 5) (Ermler et al., 1995, Ilari et al., 2002). The heme molecule that is embedded in a hydrophobic crevice formed by the 6 α-helices (Weber and Vinogradov 2001, Ilari et al., 2002, Frey and Kallio, 2003) showed a number of residues that are invariant. Overall the amino acid sequences are 44% identical and the residues that are involved in stabilization of the heme bound dioxygen (Tyr-B10, Gln-E7) are conserved. Moreover, the His-F8, Tyr-G5 and Glu-H23 residues that are conserved in filamentous fungal flavohemoglobin that are involved in formation of the catalytic triad at the proximal site (Frey and Kallio, 2003) are also conserved in Vitreosciela hemoglobin.

2.2.2 Hemoglobin Overexpression in Aspergillus oryzae Transformants

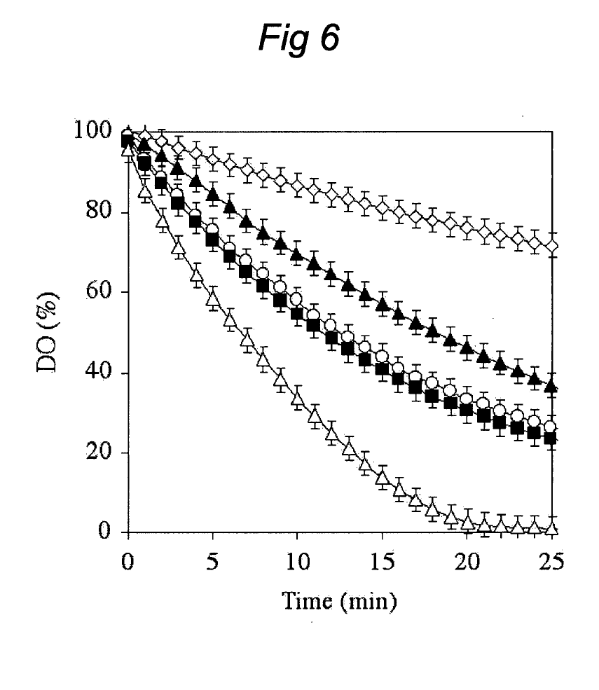

The A. oryzae and A. niger hemoglobin domain encoding genes were overexpressed in A. oryzae. One transformant of each overexpression plasmid (pHBO and pHBN, respectively) was selected for further analysis. To determine whether the transformants produced hemoglobin, cell free extracts were analyzed by SDS-PAGE. As overproduction of the 16 kDA hemoglobin domain could not be detected in the protein extracts of the transformants an alternative method to demonstrate hemoglobin overproduction was chosen. Cells were harvested after 20 hrs of growth in complete medium and transferred to oxygen saturated complete medium. The change of dissolved oxygen (DO) in the growth medium was determined after addition of either wild type or transformed cells (FIG. 6). This analysis shows that the cells overproducing the Aspergillus hemoglobins show a marked faster decrease in the amount of dissolved oxygen compared to the wild-type cells (Table 2). These results indicate that both the A. niger and A. oryzae hemoglobin domain genes are expressed in an active confirmation. Moreover, this implies that hemoglobin overproduction increases the respiratory capacity of A. oryzae.

### 2.2.3 Growth, Growth Rate and Enzyme Production in Solid State Fermentation

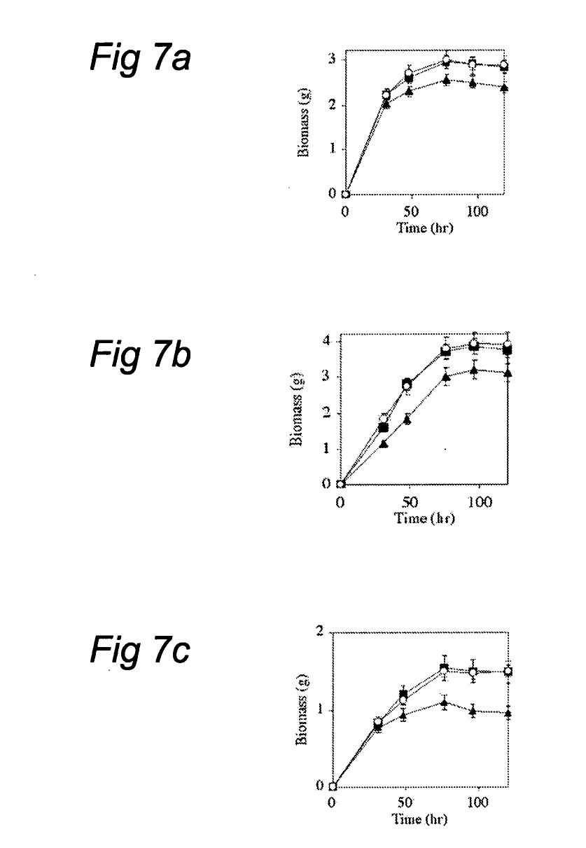

To determine the impact of overproduction of the Aspergillus hemoglobin domains on growth of A. oryzae, transformants were grown on filters that were placed on top of minimal medium (MM), potato dextrose agar (PDA) and 5% WSM. FIG. 7 shows that with the hemoglobin-producing strains, the biomass yield is significantly higher (at least 1.3 times) when grown on the different media compared to the wild-type strain. Dry weight measurements confirmed the observed difference between the hemoglobin-producing strains and the wild-type (not shown). These results suggest that the hemoglobin-producing strains have better access to the substrates compared to the wild-type.

Besides biomass weight, also different enzyme activities were measured in the extracts of the hemoglobin-producing and wild-type strains grown for 3 days on 5% WSM. Table 3 shows that the α-amylase, glucoamylase and protease activities are all higher in the hemoglobin-producing strains compared to the wild-type. In another approach the hemoglobin-producing and wild-type strains were grown for 5 and 6 days on ground wheat kernel and thereafter enzyme activities were determined in the extracts of these cultivations. Table 3 shows that the α-amylase activity in the extract of the hemoglobin-producing strains is at least 30% and 60% higher compared to that of the wild-type after respectively 5 and 6 days of growth. The glucoamylase activity is at least 9 times higher in the extracts of the hemoglobin expressing strains compared to that of the wild type strain. The protease activities measured in the extracts of the hemoglobin expressing strains are at least 3.8 and 4.5 times higher compared to that of the wild-type strain after respectively 5 and 6 days of growth on the ground wheat kernel.

### 2.2.4 Growth, Growth Rate and Enzyme Production in Submerged Fermentation

Selected A. niger pHBN transformants from a laccase producing transformant (Record et al., 2002, Eur. J. Biochem. 269: 602-9) were cultivated in shake flasks with complex growth medium which due to culture viscosity would result in O2 limitation. Culture pH and glucose consumption were used as measures to compare kinetics of the various fermentations, biomass, total secreted protein and laccase production were used to determine the effect of flavohemoglobin production. The pH profile and the glucose consumption profiles were almost identical showing complete glucose consumption after 2 days of culture of the parental and transformant strains (see Table 5).

The analysis of the total biomass production and of laccase production in Table 5 showed that in particular in a pHBN transformant (strain Hb-niger#02) both a twofold higher biomass levels and twofold more laccase was observed. Specific laccase productivity (per mg total protein) was even more than 5-fold higher than in the parental strain. Also Hb-niger#05 produced more laccase.

TABLE 1
Fungal species with database accession numbers of which the flavoHb
protein sequences are used in this study. The identities are determined
after comparison to the A. niger flavoHb protein sequence.
Abbreviations are the same as in FIG. 1 and 2. Database numbers were
obtained from NCBI (www.ncbi.nlm.nih.gov/) or in case of Afu, Pan
(1 &2) and Pch from (//www.tigr.org), (//podospora.igmors.u-psud.fr/)
and (//www.jgi.doe.gov/), respectively.

| | Abbre- | | Identity |
|---|---|---|---|
| Organism | viation | Database number | (%) |
| Aspergillus niger | Anr | CAF25490.1 | 100 |
| Aspergillus fumigatus | Afu | TIGR_5085contig5277 | 65 |
| Aspergillus nidulans | Ans1 | EAA59083.1 | 58 |
| Podospora anserina | Pan1 | Contig430 | 46 |
| Podospora anserina | Pan2 | Contig132 | 46 |
| Gibberella zeae | Gze2 | EAA70711.1 | 45 |
| Aspergillus nidulans | Ans2 | EAA61421.1 | 45 |
| Gibberella zeae | Gze1 | EAA73242.1 | 44 |
| Neurospora crassa | Ncr | XP_32928.2 | 43 |
| Neurospora crassa | Ncr | XP_323418.1 | 43 |
| Cryptococcus neoformans | Cne | EAL22289.1 | 43 |
| Fusarium oxysporum | Fox | BGA33011.1 | 43 |
| Magnaporthe grisea | Mgr | EAA48540.1 | 43 |
| Phanerochaete | Pch | AADS01000126.1 | 42 |
| chrysosporium | | | |
| Alcaligenus eutrophus | Aeu | A53396 | 42 |
| Eschericia coli | Eco | BAA16460 | 37 |
| Saccharomyces cerevisiae | Sce | NC_001139.2 | 37 |
| Candida glabrata | Cgl | CAG62036.1 | 35 |
| Kluyveromyces lactis | Kla | CAH02568.1 | 35 |
| Deboramyces hansenii | Dha1 | XP_462620 | 33 |
| Schizosaccharomyces | Spo | NC_003424.1 | 32 |
| pombe | | | |
| Deboramyces hansenii | Dha2 | XP_462633.1 | 32 |
| Yarrowia lipolytica | Yli | XP_502088 | 30 |
| Candida albicans | Cal3 | EAK91821.1 | 29 |
| Pichia norvegensis | Pno | S26964 | 29 |
| Candida albicans | Cal2 | EAK91824.1 | 27 |
| Candida albicans | Cal1 | EAL00511.1 | 26 |

TABLE 2
Oxygen consumption, growth rates and enzyme activities of the hemoglobin-
producing and wild-type strains. Oxygen consumption rates were determined from
results shown in FIG. 6 presuming that the oxygen consumption was constant during
the first 2 minutes and expressed in decreased percentage per minute (% * min⁻¹).
Growth rates were determined from the results in FIG. 3B presuming that they were
constant during the first 30 hours of growth on 5% WSM and were expressed in amount
of wet weight biomass formed per hr (mg/hr). Enzyme activities measured in extracts
after 3 days of growth of the wildtype, and the hemoglobin-producing strains
(harboring plasmid pHBN and pHBO) grown on 5% WSM. Extracts were prepared as
described (te Biesebeke et al., 2004). Enzyme activities were expressed per amount of
wet weight solid-state fermentation sample (U/mg).

| | O₂ | Growth | | | Protease | Protease | Protease |
|---|---|---|---|---|---|---|---|
| A. oryzae | consumption | Rate | Amylase | Glucoamylase | pH 5.5 | pH 7 | pH 8.5 |
| Strain | (% * min⁻¹) | (mg/hr) | (U/mg) | (U/mg) | (U/mg) | (U/mg) | (U/mg) |
| WT | 3.4 | 37 | 952 | 0 | 95 | 148 | 667 |
| PHBN | 5.1 | 51 | 1524 | 11 | 214 | 233 | 1095 |
| PHBO | 5.0 | 58 | 1773 | 9 | 190 | 281 | 1238 |

TABLE 3
Enzyme activities measured in extracts after 5 and 6 days (D) of growth
of the wild-type, the A. niger (pHBN) and A. oryzae (pHBO)
expressing hemoglobin strains grown on ground wheat kernels. Extracts
were prepared as described (te Biesebeke et al., 2004). The results are
the average of 2 experiments. Standard deviations did not exceed 13%
of the shown values.

| A. | | Amyl- | | Protease | Protease | Protease |
|---|---|---|---|---|---|---|
| oryzae | Time | ase | Glucoamylase | PH 5.5 | pH 7 | pH 8.5 |
| Strain | (days) | (U/g) | (U/g) | (U/g) | (U/g) | (U/g) |
| WT | 5 | 129 | 0.8 | 12 | 10 | 13 |
| PHBN | 5 | 168 | 7.4 | 53 | 57 | 47 |
| PHBO | 5 | 200 | 12.8 | 59 | 64 | 48 |
| WT | 6 | 134 | 1.0 | 14 | 16 | 14 |
| PHBN | 6 | 214 | 12.9 | 88 | 96 | 61 |
| PHBO | 6 | 229 | 11.7 | 91 | 109 | 83 |

TABLE 4
Amino acid sequence identities of Hemoglobin
domains of flavoHb proteins listed in Table 1.

| | Abbre- | | Identity |
|---|---|---|---|
| Organism | viation | Database number | (%) |
| Aspergillus oryzae | Aor | CAF32307.1 | 100 |
| Aspergillus fumigatus | Afu | TIGR_5085contig5277 | 82 |
| Aspergillus nidulans | Ans2 | EAA61421.1 | 80 |
| Aspergillus niger | Anr | CAF25490.1 | 71 |
| Magnaporthe grisea | Mgr | EAA48540.1 | 57 |
| Podospora anserine | Pan2 | Contig132 | 56 |
| Aspergillus nidulans | Ans1 | EAA59083.1 | 56 |
| Gibberella zeae | Gze1 | EAA73242.1 | 55 |
| Fusarium oxysporum | Fox | BGA33011.1 | 55 |
| Cryptococcus neoformans | Cne | EAL22289.1 | 54 |
| Candida glabrata | Cgl | CAG62036.1 | 54 |
| Podospora anserine | Pan1 | Contig430 | 53 |
| Neurospora crassa | Ncr | EAA34752.1 | 53 |
| Neurospora crassa | Ncr | EAA28703.1 | 52 |
| Gibberella zeae | Gze2 | EAA70711.1 | 51 |
| Saccharomyces cerevisiae | Sce | NC_001139.2 | 51 |
| Vitreoscilla sp. C1 | VC1 | AAA7506 | 48 |
| Alcaligenus eutrophus | Aeu | A53396 | 48 |
| Eschericia coli | Eco | BAA16460 | 47 |
| Deboramyces hansenii | Dha1 | CAG91139.1 | 47 |
| Vitreoscilla stercoraria | Vst | AAT01097.1 | 45 |
| Phanerochaete | Pch | AADS01000126.1 | 43 |
| chrysosporium | | | |
| Deboramyces hansenii | Dha2 | CAG91152.1 | 43 |
| Schizosaccharomyces | Spo | NC_003424.1 | 42 |
| pombe | | | |
| Candida albicans | Cal3 | EAK91821.1 | 39 |
| Pichia norvegensis | Pno | S26964 | 37 |
| Candida albicans | Cal2 | EAK91824.1 | 34 |
| Kluyveromyces lactis | Kla | CAH02568.1 | 27 |
| Candida albicans | Cal1 | EAL00511.1 | 26 |
| Yarrowia lipolytica | Yli | CAG81069.1 | 23 |

TABLE 5
Submerged fermentations with laccase producing A. niger
pHBN transformants (see Example 2.2.4).

| | 0 hr | 24 hrs | 48 hrs | 69 hrs | Day 4 |
|---|---|---|---|---|---|
| pH culture medium | | | | | |
| Laccase | 6.7 | 6.24 | 6.72 | 7.47 | 7.76 |
| HB #2 | 6.7 | 5.05 | 6.24 | 6.88 | 7.27 |
| HB #5 | 6.7 | 6.77 | 6.93 | 7.67 | 7.85 |
| Biomass (g/culture) | | | | | |
| Parent strain | 0 | 0.052 | 0.224 | 0.274 | 0.1 |
| HB #2 | 0 | 0.06 | 0.188 | 0.2 | 0.1 |
| HB #5 | 0 | 0.07 | 0.172 | 0.165 | 0.1 |
| Laccase (Units/ml) | | | | | |
| Parent strain | 0 | 0 | 0 | 0.02 | 0.08 |
| HB #2 | 0 | 0 | 0.01 | 0.05 | 0.13 |
| HB #5 | 0 | 0.3 | 0.01 | 0.07 | 0.12 |
| Extracell. Protein | | | | | |
| (mg/ml) | | | | | |
| Parent strain | 0 | 0 | 78 | 249 | — |
| HB #2 | 0 | | 14 | 62 | 125 |
| HB #5 | 0 | 4 | 62 | 109 | 108 |

### REFERENCES

Altschul S F., Madden T L, Schäffer A A, Zhang J, Zhang Z, Miller W, Lipman D J. (1997) Gapped BLAST and PSI-BLAST: a new generation of protein database search programs. Nucleic Acids Res 25:3389-3402.
Andersson C I, Arfvidsson C, Kallio P T, Wahlund K G, Bulow L. (2003) Enhanced ribosome and tRNA contents in Escherichia coli expressing a truncated Vitreoscilla hemoglobin mutant analyzed by flow field-flow fractionation. Biotechnol Lett 25:1499-504.
Bhave S L, Chattoo B B. (2002) Expression of vitreoscilla hemoglobin improves growth and levels of extracellular enzyme in Yarrowia lipolytica. Biotechnol Bioeng 84:658-666.
Buisson N, Labbe-Bois R. (1998) Flavohemoglobin expression and function in Saccharomyces cerevisiae. No relationship with respiration and complex response to oxidative stress. J Biol Chem 273:9527-9533.
Crawford M J, Sherman D R, Goldberg D E. (1995) Regulation of Saccharomyces cerevisiae Flavohemoglobin gene expression. J Biol Chem 270: 6991-6996.
Demodena J A, S Gutierrez, J Velasco, F J Fernandez, R A Fachini, J L Galazo, D E Hughes, J F Martin. (1993) The production of Cephalosporin C by Acremonium chrysogenum is improved by the intracellular expression of a bacterial hemoglobin. Biotechnol 11:926-929.

Ermler U, Siddiqui R A, Cramm R, Friedrich B. (1995) Crystal structure of the flavohemoglobin from Alcaligenes eutropus at 1.75 A resolution. EMBO J 14:6067-6077.
Frey A D, Kallio P T. (2003) Bacterial hemoglobins and flavohemoglobins:versatile proteins and their impact on microbiology and biotechnology. FEMS Microbiology reviews 27: 525-545.
Gasch A P, Spellman P T, Kao C M, Carmel-Harel O, Eisen M B, Storz G, Botstein D, Brown P O. (2000) Genomic expression programs in the response of yeast cells to environmental changes. Mol Biol Cell 11:4241-4257.
Geckil H, Gencer S, Kahraman H, Erenler S O. (2003) Genetic engineering of Enterobacter aerogenes with the Vitreoscilla hemoglobin gene: cell growth, survival, and antioxidant enzyme status under oxidative stress. Res Microbiol 154:425-431.
Ilari, A., A. Bonamore, A. Farina, K. A. Johnson, A. Boffi. (2002) The X-ray structure of ferric Escherichia coli flavohemoglobin reveals an unexpected geometry of the distal heme pocket. J Biol Chem 277:23725-223732.
Howley P M, Israel M F, Law M F, Martin M A. (1979) A rapid method for detecting and mapping homology between heterologous DNAs. J Biol Chem 254:4876-4883.
Jesus-Berrios M, Liu L, Nussbaum J C, Cox G M, Stamler J S, Heitman J. (2003) Enzymes that counteract nitrosative stress promote fungal virulence. Curr Biol 13:1963-1968.
Kobayashi G, Nakamura T, Ohmachi H, Matsuoka A, Ochiai T, Shikama K. (2002) Yeast flavohemoglobin from Candida norvegensis. Its structural, spectral, and stability properties. J Biol Chem 277:42540-42548.
Lin Y H, Li Y F, Huang M C, Tsai Y C. (2004) Intracellular expression of Vitreoscilla hemoglobin in Aspergillus terreus to alleviate the effect of a short break in aeration during culture. Biotechnol Lett 26:1067-1072.
Liu C, He Y, Chang Z. (2004) Truncated hemoglobin o of Mycobacterium tuberculosis: the oligomeric state change and the interaction with membrane components. Biochem Biophys Res Commun 316(4):1163-1172.
Mizutani O, Nojima A, Yamamoto M, Furukawa K, Fujioka T, Yamagata Y, Abe K, Nakajima T. (2004) Disordered cell integrity signaling caused by disruption of the kexB gene in Aspergillus oryzae. Euc Cell 3:1036-1048.
Nantel A, Dignard D, Bachewich C, Harcus D, Marcil A, Bouin A P, Sensen C W, Hogues H, van het Hoog M, Gordon P, Rigby T, Benoit F, Tessier D C, Thomas D Y, Whiteway M. (2002) Transcription profiling of Candida albicans cells undergoing the yeast-to-hyphal transition. Mol Biol Cell 13:3452-3465.
Oostra, J, le Comte E P, van den Heuvel J C, Tramper J, Rinzema A. (2001) Intra-particle oxygen diffusion limitation in solid-state fermentation. Biotechnol Bioeng 75:13-24.
Rahardjo Y S, Weber F J, le Compte E P, Tramper J, Rinzema A. (2001) Contribution of aerial hyphae of Aspergillus oryzae to respiration in a model solid state fermentation. Biotechnol Bioeng 78:539-544.
Saitou N, Nei M. (1987) The neighbor joining method: a new method for reconstructing phylogenetic trees. Mol Biol Evol 4:406-425.
Takaya N, Suzuki S, Matsuo M, Shoun H. (1997) Purification and characterization of a flavohemoglobin from the denitrifying fungus Fusarium oxysporum. FEBS Lett 414:545-548.
Tatusova T A, T L Madden. (1999) Blast 2sequences—a new tool for comparing protein and nucleotide sequences. FEMS Microbiol Lett 174: 247-250.
Te Biesebeke R, Levin A, Sagt C, Bartels J, Goosen T, Ram A F, Punt P J, Cees van den Hondel CAMJJ. (2005) Identification of growth phenotype related genes of Aspergillus oryzae by using heterologous macro array analysis and suppression subtractive hybridisation. Molecular Genetics Genomics 272 (6). [Epub ahead of print]
Te Biesebeke R, Ruijter J G, Rahardjo S P, Hoogschagen M J, Heerikhuisen M, Levin A, van Driel K G A, Schutyser M A I, Dijksterhuis J, Zhu Y, Weber F J, de Vos W M, van den Hondel CAMJJ, Rinzema A, Punt. P J (2002) Aspergillus oryzae in solid state and submerged fermentations. FEMS yeast research 2:245-248.
Te Biesebeke R, van Biezen N, de Vos W M, van den Hondel CAMJJ, Punt P J. (2004) Different control mechanisms regulate the glucoamylase and protease gene transcription in Aspergillus oryzae in Solid-state and Submerged Fermentation. Appl Microbiol Biotechnol. 2004 Dec. 3; [Epub ahead of print]
Thibault J., Pouliot K., Agosin A. and Pérez-Correa R.(2000), “Reassessment of the Estimation of Dissolved Oxygen Concentration Profile and KLa in Solid-State Fermentation”, Process Biochem. 36, 9-18.
Thomson J D, Higgins D G, Gibson T J. (1994) CLUSTAL W: improving the sensitivity of progressive multiple sequence alignment through sequence weighting, position-specific gap penalties and weight matrix choice. Nucl Acids Res 22:4773-4680.
Ullmann B D, Myers H, Chiranand W, Lazzell A L, Zhao Q, Vega L A, Lopez-Ribot J L, Gardner P R, Gustin M C. (2004) Inducible defense mechanism against nitric oxide in Candida albicans. Eukaryot Cell 3:715-723.
Van den Hondel CAMJJ, Punt P J, van Gorcum R F M. (1992) Production of extracellular proteins by the filamentous fungus Aspergillus. Antonie van Leeuwenhoek 61: 153-160.
van Hartingsveldt W, Mattern I E, van Zeijl C M, Pouwels P H, van den Hondel C A. (1987) Development of a homologous transformation system for Aspergillus niger based on the pyrG gene. Mol Gen Genet. 206:71-75.
Veldhuisen G, Saloheimo M, Fiers M A, Punt P J, Contreras R, Penttila M, van den Hondel CAMJJ. (1997) Isolation and analysis of functional homologues of secretion-related SAR1 gene of Saccharomyces cerevisiae from Aspergillus niger and Trichoderma reesei. Mol Gen Genet. 256:446-455.
Verdoes J C, Punt P J, Schrickx J M, van Verseveld H W, Stouthamer A H, van den Hondel CAMJJ. (1993) Glucoamylase overexpression in Aspergillus niger: molecular genetic analysis of strains containing multiple copies of the glaA gene. Transgenic Res. 2:84-92.
Weber R E, Vinogradov S N. (2001) Nonvertebrate hemoglobins: functions and molecular adaptations. Phys rev 8: 569-628.
Webster and Liu, (1974) Reduced nicotinamide adenine dinucleotide cytochrome reductase associated with cytochrome o purified from Vitreoscilla. Evidence for an intermediate oxygenated form of cytochrome o. J Biol. Chem. 249: 4257-60.
Wu J M, Hsu T A, Lee C K. (2003) Expression of the gene coding for bacterial hemoglobin improves beta-galactosidase production in a recombinant Pichia pastoris. Biotechnol Lett 25:1457-1462.
Yolon A A, Shabarova Z A (1990) Constructing DNA by polymerase recombination. Nucleic acids Res. 18:3983-3986.
Yon J, Fried M (1989) Precise gene fusion by PCR. Nucleic Acids Res 17:4895.
Yu H, Shi Y, Zhang Y, Yang S, Shen Z. (2002) Effect of Vitreoscilla hemoglobin biosynthesis in Escherichia coli on production of poly(beta-hydroxybutyrate) and fermentative parameters. FEMS Microbiol Lett 214:223-227.
Zhao X J, Raitt D, V Burke P, Clewell A S, Kwast K E, Poyton R O. (1996) Function and expression of flavohemoglobin in Saccharomyces cerevisiae. Evidence for a role in the oxidative stress response. J Biol Chem 271:25131-25138.
Zhu H, A F Riggs (1992) Yeast flavohemoglobin is an ancient protein related to globins and a reductase family. Proc Natl Acad Sci USA 89:5015-5019.

1. A fungal host cell transformed with a nucleic acid construct comprising a nucleotide sequence encoding a fungal oxygen-binding protein or a fragment thereof that comprises an oxygen-binding domain, wherein the nucleic acid construct upon transformation of the host cell, confers to the host cell an increase in a fermentation parameter compared to an otherwise identical host cell that is not transformed with the construct, whereby the fermentation parameter is at least one of:
a) oxygen uptake rate;
b) biomass density;
c) volumetric productivity; and,
d) yield coefficient of fermentation product produced over substrate.

2. A host cell according to claim 1, wherein the fermentation parameter of the transformed host cell is increased by at least 5% as compared to the untransformed host cell.

3. A host cell according to claim 1, wherein oxygen-binding protein is a flavohemoglobin or wherein the oxygen-binding domain is a hemoglobin domain.

4. A host cell according to claim 1, wherein the nucleotide sequence is selected from the group consisting of:
(a) nucleotide sequences encoding a polypeptide comprising an amino acid sequence that has at least 49% sequence identity with the amino acid sequence of SEQ ID NO. 1 or 2;
(b) nucleotide sequences the complementary strand of which hybridises to a nucleic acid molecule sequence of (a); and,
(c) nucleotide sequences the sequence of which differs from the sequence of a nucleic acid molecule of (b) due to the degeneracy of the genetic code.

5. A host cell according to claim 1, wherein the nucleotide sequence encodes an amino acid sequence that has at least 90% amino acid identity with the amino acid sequence of a fungal flavohemoglobin that naturally occurs in the host or with the amino acid sequence of a fragment of the flavohemoglobin comprising the hemoglobin domain.

6. A host cell according to claim 1, wherein the fragment comprising the hemoglobin domain comprises no more than 30, 15, 8, or 4 additional amino acids onto either terminus of the domain, whereby the domain is defined as a polypeptide consisting of an amino acid sequence that has at least 49% sequence identity with the amino acid sequence of SEQ ID NO. 1 or 2.

7. A host cell according to claim 1, wherein the host cell is a filamentous fungus that belongs to one of the genera: Aspergillus, Trichoderma, Humicola, Acremonium, Fusarium, Rhizopus, Mortierella, Penicillium, Myceliophthora, Chrysosporium, Mucor, Sordaria, Neurospora, Podospora, Monascus, Agaricus, Pycnoporus, Schizophylum, Trametes and Phanerochaete.

8. A host cell according to claim 1, wherein the host cell is a yeast that belongs to one of the genera: Saccharomyces, Kluyveromyces, Candida, Pichia, Schizosaccharomyces, Hansenula, Kloeckera, Schwanniomyces, and Yarrowia.

9. A process for producing a fermentation product, wherein the process comprises conversion of a substrate by a transformed host cell as defined in claim 1 into the fermentation product.

10. A process according to claim 9, wherein the process is an aerobic fermentation process.

11. A process according to claim 9, wherein one or more of the following fermentation parameters of the process with the transformed host cell is at least 5% higher than in an otherwise identical process with the untransformed host cell:
(a) oxygen uptake rate;
(b) biomass density;
(c) volumetric productivity; and,
(d) yield coefficient of fermentation product produced over substrate.

12. A process according to claim 9, wherein the process is a solid state fermentation process.

13. A process according to claim 9, wherein the fermentation product is selected from biomass comprising the host cell, a primary metabolite, secondary metabolite or a peptide.

14. A process according to claim 13, wherein the fermentation product is an organic compound selected from glucaric acid, gluconic acid, glutaric acid, adipic acid, succinic acid, tartaric acid, oxalic acid, acetic acid, lactic acid, formic acid, malic acid, maleic acid, malonic acid, citric acid, fumaric acid, itaconic acid, levulinic acid, xylonic acid, aconitic acid, ascorbic acid, kojic acid, comeric acid, an amino acid, a poly unsaturated fatty acid, ethanol, 1,3-propane-diol, ethylene, glycerol, xylitol, carotene, astaxanthin, lycopene, and lutein.

15. A process according to claim 13, wherein the fermentation product is a β-lactam antibiotic, a cephalosporin, cyclosporin or lovastatin.

16. A process according to claim 13, wherein the fermentation product is a peptide selected from an oligopeptide, a polypeptide, a (pharmaceutical or industrial) protein and an enzyme.

17. A process according to claim 16, the peptide is secreted from the host cell, preferably into the culture medium.

18. An isolated nucleic acid molecule comprising a nucleotide sequence encoding an oxygen-binding protein, whereby the nucleotide sequence is selected from:
(a) nucleotide sequences encoding a polypeptide comprising an amino acid sequence that has at least 66% sequence identity with the amino acid sequence of SEQ ID NO. 3;
(b) nucleotide sequences the complementary strand of which hybridises to a nucleotide sequence of (a); and,
(c) nucleotide sequences the sequence of which differs from the sequence of a nucleotide sequence of (b) due to the degeneracy of the genetic code.

19. An isolated nucleic acid molecule comprising a nucleotide sequence encoding an oxygen-binding protein, whereby the nucleotide sequence is selected from:
(a) nucleotide sequences encoding a polypeptide comprising an amino acid sequence that has at least 78% sequence identity with the amino acid sequence of SEQ ID NO. 2;
(b) nucleotide sequences the complementary strand of which hybridises to a nucleotide sequence of (a); and,
(c) nucleotide sequences the sequence of which differs from the sequence of a nucleotide sequence of (b) due to the degeneracy of the genetic code.

20. An isolated nucleic acid molecule comprising a nucleotide sequence encoding an oxygen-binding protein, whereby the nucleotide sequence is selected from:
(a) nucleotide sequences encoding a polypeptide comprising an amino acid sequence that has at least 83% sequence identity with the amino acid sequence of SEQ ID NO. 1;
(b) nucleotide sequences the complementary strand of which hybridises to a nucleotide sequence of (a); and,
(c) nucleotide sequences the sequence of which differs from the sequence of a nucleotide sequence of (b) due to the degeneracy of the genetic code.

21. An isolated nucleic acid molecule according to claim 18, wherein the nucleotide sequence when present in an expression construct upon transformation of a fungal host cell, confers to the host cell an increase in a fermentation parameter compared to an otherwise identical host cell that is not transformed with the construct, whereby the fermentation parameter is at least one of:
(a) oxygen uptake rate;
(b) biomass density;
(c) volumetric productivity; and,
(d) yield coefficient of fermentation product produced over substrate.

22. An isolated polypeptide comprising an amino acid sequence selected from:
(a) amino acids sequences that have at least 66% sequence identity with the amino acid sequence of SEQ ID NO. 3;
(b) amino acids sequences that have at least 78% sequence identity with the amino acid sequence of SEQ ID NO. 2; and,
(c) amino acids sequences that have at least 83% sequence identity with the amino acid sequence of SEQ ID NO. 1.

23. An isolated polypeptide according to claim 22, wherein the polypeptide when expressed in a fungal host cell from an expression construct comprising a nucleotide sequence encoding the polypeptide, upon transformation of the host cell with the expression construct, confers to the host cell an increase in a fermentation parameter compared to an otherwise identical host cell that is not transformed with the construct, whereby the fermentation parameter is at least one of:
(a) oxygen uptake rate;
(b) biomass density;
(c) volumetric productivity; and,
(d) yield coefficient of fermentation product produced over substrate.
